(19) United States
(12) Patent Application Publication
(10) Pub. No.: US 2025/0085284 A1
(43) Pub. Date: Mar. 13, 2025

(54) PRODUCTS AND USES THEREOF FOR PREDICTING THE SENSITIVITY OF A SUBJECT TO CANCER IMMUNOTHERAPY INVOLVING AN ANTI-PD(L)1 AND AN ANTI-ANGIOGENIC AGENT, AND FOR SELECTING OPTIMIZED THERAPY

(71) Applicants: INSTITUT GUSTAVE ROUSSY, VILLEJUIF (FR); UNIVERSITE PARIS SACLAY, GIF-SUR-YVETTE (FR); INSTITUT NATIONAL DE LA SANTE ET DE LA RECHERCHE MEDICALE, PARIS (FR)

(72) Inventors: FRANÇOIS-XAVIER DANLOS, MONTREUIL (FR); AURÉLIEN MARABELLE, SCEAUX (FR); SÉVERINE MOURAUD, CLAMART (FR); NATHALIE CHAPUT-GRAS, PARIS (FR); LAMBROS TSELIKAS, CHARENTON LE PONT (FR); BASTIEN JOB, SAINT-MAURICE (FR)

(21) Appl. No.: 18/463,512

(22) Filed: Sep. 8, 2023

(51) Int. Cl. G01N 33/574; G01N 33/50; G01N 33/68; G01N 33/74

(52) U.S. Cl. CPC G01N 33/574; G01N 33/505; G01N 33/6869; G01N 33/74; G01N 2333/475; G01N 2333/5412; G01N 2333/5421; G01N 2333/96436; G01N 2800/52

(57) ABSTRACT

The present invention relates to a method of predicting assessing or monitoring the sensitivity of a subject having a cancer to a combination therapy, preferably to a therapy combining an immunotherapeutic agent and an anti-angiogenic agent, and to corresponding kits and uses thereof. The method of predicting, assessing or monitoring the sensitivity of a subject having a cancer or malignant tumor to a proposed combination therapy typically comprises a step a) of determining, in a biological sample from said subject, the presence, absence or expression level or proportion of at least one biomarker, for example at least two biomarkers, and when the expression level or proportion is determined a step b) of comparing said expression level or proportion to reference expression level(s) or to reference expression ratio(s), thereby predicting, assessing or monitoring whether the subject having a tumor is responsive or resistant to the proposed combination therapy.

### FIELD OF THE INVENTION

The present invention relates to a method of predicting, assessing or monitoring the sensitivity of a subject having a cancer or malignant tumor to a combination therapy, and to corresponding kits. The method of predicting, assessing or monitoring the sensitivity of a subject having a cancer or malignant tumor to a proposed combination therapy typically comprises a step a) of determining, in a biological sample from said subject, the presence, absence or expression level or proportion of at least one biomarker, for example at least two biomarkers, and when the expression level or proportion is determined a step b) of comparing said expression level or proportion to a reference expression level or to a reference expression ratio, thereby predicting, assessing or monitoring whether the subject having a tumor is responsive or resistant to the proposed combination therapy.

### BACKGROUND OF THE INVENTION

Unresectable malignant mesothelioma (MM) is a cancer developed from pleural and peritoneal serous membrane linings, upon chronic exposure to environmental silicate minerals such as asbestos (Galateau-Salle F. et al.). Indeed, chronic exposure of serous membranes to asbestos microparticles leads to inflammation, recruitment of inflammatory macrophages, development of an immunosuppressive pro tumoral microenvironment, constitution of pathological neoangiogenesis with hypoxia and eventually serous cells malignant transformation toward an aggressive phenotype leading to metastatic disease (Xu A. et al.; Dostert C. et al.). Advanced MM are incurable cancers with historical median overall survival typically around 12 months with platinum doublet chemotherapies (Vogelzang N J et al.) but slightly better for epithelioid than non-epithelioid (biphasic & sarcomatoid) histotypes (Musk A W et al.). Targeting the tumor microenvironment (TME) with immune checkpoint blockers or antiangiogenic drugs, has shown recently significant activity in several metastatic cancers. Angiogenesis contributes to tumor growth and the development of metastases but the modulation of neo-angiogenesis via inhibition of the VEGF/VEGFR pathway has shown anti-tumor activity in several human solid cancers (Grothey A. et al.). Also, blocking immune checkpoints with antagonistic monoclonal antibodies targeting Programmed death-1 receptor (PD-1) and its ligand (PD-L1) have been extensively investigated in the recent past and are still in active development across malignancies (Ribas A. et al.). Combining anti-angiogenic drugs and anti-PD-(L)1 antibodies has recently shown important synergistic results in renal cell carcinoma (RCC), hepatocellular carcinoma (HCC) and non-small cell lung carcinoma (NSCLC) (Finn R S et al.; Rini B I et al.; Socinski M A et al.). Indeed, anti-VEGF therapies may enhance anti PD-1/PD-L1 efficacy by reversing VEGF-mediated immunosuppression and promote T-cell infiltration in tumors (Wallin J J et al.). Recently, translational studies have illustrated that the PD-1/PD-L1 and angiogenesis pathways were involved in MM tumors and could better characterize the biology of those tumors than the historical histotypes (Alcala N. et al.). Both anti-angiogenic and immune checkpoint blockers have indeed shown activity in advanced pleural mesothelioma. Bevacizumab, an IgG1 monoclonal antibody targeting VEGFA, in association with platinum-based regimen chemotherapy, has been shown to increase the overall survival of patients with untreated unresectable pleural mesothelioma, compared to chemotherapy alone (Zalenman G. et al.). Nintedanib, an oral triple receptor tyrosine kinase inhibitor (TKI) of PDGFRα/β, FGFR1-3, and VEGFR1-3, has shown significant activity in combination with chemotherapy for the treatment of pleural MM in a randomized phase 2 trial (Grosso F. et al.) but this result could not be confirmed in a subsequent Phase 3 trial (Scagliotti G V et al.). Pembrolizumab, an IgG4 monoclonal anti-PD1 antibody, has shown limited activity in monotherapy for advanced mesothelioma with an objective response rate (ORR) of 8% (Yap T A et al.). More recently, the combination of an anti-PD-L1 (atezolizumab) with an anti-VEGF (bevacizumab) exhibited significant synergistic activity with an ORR of 40% and a median duration or response of 12.8 months in peritoneal mesothelioma (Raghav K. et al.).

To date, the combination of anti PD-1 (Nivolumab) and anti CTLA-4 (Ipilimumab) antibodies (anti PD-1+anti CTLA-4) has demonstrated to improve significantly the OS of MM patients with untreated unresectable pleural mesothelioma, compared to standard of care chemotherapy (Baas P. et al.). This combined immunotherapy had an ORR comparable to chemotherapy (˜40%) in both epithelioid and non-epithelioid histotypes. Chemotherapy was known to provide better outcomes in epithelioid rather than sarcomatoid mesotheliomas (Musk A W et al.). Interestingly, mesothelioma histology is not impacting the efficacy of an anti-PD1 and anti-CTLA4 combination (Baas P. et al.). Therefore, the biggest overall survival benefit of anti-PD1 and anti-CTLA4 in comparison with chemotherapy is for sarcomatoid mesothelioma. The median duration of response was longer for immunotherapy (11 months) than for chemotherapy (6.7 months) which translated into a significant benefit in OS (median OS of 18.1 vs 14.1 months).

Therefore, it has become clear that immune checkpoint targeted immunotherapies mostly benefit to MM patients who are responding to such treatments and that therapeutic improvements remain to be done for the majority of patients that are not benefiting from them. However, little is currently known about the biology of MM tumors presenting with primary resistance to anti-PD(L)1 based therapies. In the above mentioned Nivolumab+Ipilimumab trial, a slight decrease in efficacy was found when MM tumor cells did not express PD-L1 (<1% PD-L1 expression by 28-8 IHC staining on FFPE samples) (Baas P. et al.). The ancillary analysis of the above mentioned Atezolizumab+Bevacizumab trial could not identify significant biomarkers of activity besides an epithelial-to-mesenchymal (EMT) gene expression signature in tumors not responding to the combination therapy (Raghav K. et al.). Of note, the ETOP Beat Meso trial is an ongoing multicentre randomized phase 3 study currently testing the value of adding an anti-PD-L1 (atezolizumab) to a combination of anti-VEGF (bevacizumab) and standard chemotherapy (NCT03762018). This trial follows the good results obtained by the two Phase 2 trials DREAM (ACTRN12616001170415) and PrE0505 (NCT02899195) testing an anti-PD-L1 (durvalumab) in combination with cisplatin and pemetrexed with response rates of 46% and 56% respectively in first line advanced pleural mesothelioma (Nowak A K et al.; Forde P M et al.).

Here, inventors report the safety and efficacy of nintedanib, a pan anti-angiogenic TKI, in combination with pembrolizumab, an IgG4 monoclonal anti-PD1 antibody, in patients with advanced mesothelioma naïve to immunotherapy, and previously treated by at least one line of platinum-based chemotherapy regimen (PEMBIB trial; NCT02856425). Inventors aimed to better describe the biological correlates of primary resistance to anti-PD(L)1+anti-angiogenics in patients with pleural MM. Inventors performed an extensive exploration of blood and tumor samples at baseline and on-treatment, including the analysis of fresh blood and of fresh tumor samples by flow cytometry and by titration of cytokines, chemokines, VEGF, and soluble factors released by those fresh tissues. Their findings on fresh MM tissue, as well as fixed/frozen tissues, highlighted novel mechanisms of immunotherapy resistance, providing the rationale for the present invention allowing novel biology-driven immunotherapy strategies in the treatment of cancer, for example of MM, in particular pleural MM.

Inventors now advantageously herein provide a new diagnostic tool to identify patients suffering of cancer who can respond, i.e. actually benefit, from combined therapies, in particular from the combination of an anti-PD1 (or anti-PDL1) agent and an anti-angiogenic agent, for example from a pembrolizumab (“Pembro”) and nintendanib combination therapy.

### SUMMARY OF THE INVENTION

Inventors herein describe an in vivo, in vitro or ex vivo method of predicting, assessing or monitoring the sensitivity or resistance of a subject having a cancer to a therapy combining i) an immunotherapeutic agent, in particular an anti-PD-1, or anti-PD-L1, monoclonal antibody, and ii) an anti-angiogenic agent, after one or several treatment steps in the subject.

The method in particular comprises:
a step a) of determining in a tumor sample of the subject, the proportion of CD8+ T-cells among live CD45+ CD3+ T cells, and a step b) of comparing said live CD8+CD3+CD45+ T cells proportion to a CD8+CD3+CD45+ T cells reference proportion, a proportion of live CD8+CD3+CD45+ T cells below (<) the CD8+CD3+CD45+ T cells reference proportion being indicative of resistance of the subject to the combination therapy and a proportion of CD8+CD3+CD45+ T cells superior or equal to (≥) the CD8+CD3+CD45+ T cells reference proportion being indicative of sensitivity of the subject to the combination therapy;
a step a) of determining in a blood sample of the subject, the expression level of effector memory (EM) CD4+ T cells (i.e., CD45RA-CCR7−) expressing the alpha-4 beta-7 integrin (a4b7+ EM CD4+ T cells), cutaneous lymphocyte antigen selectin (CLA+ EM CD4+ T cells), CD49a integrin (CD49a+ EM CD4+ T cells) and/or T Helper 1 (i.e., CCR4−CCR6−CCR10−) CD4+ T-cells (Th1) expressing the CXCR3 chemokine receptor (CXCR3+ Th1 CD4+ T cells), and a step b) of comparing said expression level(s) to a4b7+ EM CD4+ T cells, CLA+ EM CD4+ T cells, CD49a+ EM CD4+ T cells and/or CXCR3+ Th1 CD4+ T cells reference expression level(s), (an) expression level(s) superior or equal to (≥) the reference expression level(s) being indicative of sensitivity of the subject to the combination therapy, and (an) expression level(s) below (<) the reference expression level(s) being indicative of resistance of the subject to the combination therapy;
a step a) of determining in a blood plasma sample or in a tumor supernatant sample of the subject, the concentration of VEGFA, VEGFD, CXCL8 and/or IL6 protein(s), and a step b) of comparing said concentration(s) to VEGFA, VEGFD, CXCL8 and/or IL6 protein(s) reference concentration(s), concentration(s) superior or equal to (≥) the reference concentration(s) being indicative of resistance of the subject to the combination therapy, and concentration(s) below the reference concentration(s) being indicative of sensitivity of the subject to the combination therapy; and/or
a step of determining in cancerous cells of a tumor sample of the subject, the number of somatic alterations, a somatic copy number alteration score or genomic instability score (“SCNA score”) above (>) a reference score being indicative of resistance of the subject to the combination therapy, and a SCNA score equal to or below (≤) the reference score being indicative of sensitivity of the subject to the combination therapy.

Also herein described is a method of selecting an appropriate therapeutic treatment for a subject having a cancer, which method comprises a step of predicting or assessing the sensitivity of a subject having a cancer to a cancer treatment combining an immunotherapeutic agent and an anti-angiogenic agent using a method of the invention.

Further herein described is a method of selecting or disqualifying a subject having a cancer for inclusion in a clinical trial, the clinical trial being for evaluating a cancer treatment combining an immunotherapeutic agent and an anti-angiogenic agent, which method comprises a step of predicting or assessing the sensitivity of a subject having a cancer to the combination therapy, using a method of the invention.

Also herein described is the use of a kit for predicting, assessing or monitoring the sensitivity of a subject having a tumor to a cancer treatment combining an anti-PD-1, or anti-PD-L1, monoclonal antibody and an anti-angiogenic agent, wherein the kit contains at least two distinct antibodies selected from an antibody recognizing a viability dye, CD45, CD8, CD3, a4b7, CLA, CD49a, CXCR3, CD45RO or CD45RA, CCR7, IL6, IL8 (CXCL8), VEGFA, VEGFD, Granzyme A, and Granzyme B, as detection means, and, optionally, a leaflet providing corresponding reference expression levels.

### DETAILED DESCRIPTION OF THE INVENTION

Cancer immunotherapy combinations have been recently shown to improve the overall survival of advanced mesotheliomas especially for patients responding to those treatments. Inventors characterized the biological correlates of malignant pleural mesotheliomas primary resistance to immunotherapy and anti-angiogenics by testing the combination of pembrolizumab, an anti-PD-1 antibody, and nintedanib, a pan anti-angiogenic tyrosine kinase inhibitor (TKI), in the multi-center PEMBIB trial (NCT02856425). Thirty patients with advanced malignant pleural mesothelioma were treated and explored. Unexpectedly, they found that refractory patients were actively recruiting CD3+CD8+ cytotoxic T-cells in their tumors through CXCL9 tumor release upon treatment. However, these patients displayed high levels of somatic copy number alterations in their tumors that correlated in particular with high blood and tumor levels of IL-6 and CXCL8. Those pro-inflammatory cytokines resulted in higher tumor secretion of VEGF and tumor enrichment in regulatory T-cells. The analysis of prospectively collected samples from patients with MM has allowed the identification of biomarkers associated with sensitivity/resistance to immunotherapy+anti-angiogenic combinations. Thanks to the present invention, advanced mesothelioma, as well as other cancers, will further benefit from stratified combination therapies adapted to their tumor biology.

By “sensitivity” or “responsiveness” is intended herein the likelihood that a patient will respond to an anti-cancer treatment (“cancer treatment”) as herein described.

By “resistant” is intended herein the likelihood that a patient will not respond to such a cancer treatment.

Predictive methods of the invention can advantageously be used clinically to make treatment decisions by choosing as soon as possible the most appropriate treatment modalities for a particular patient and limit toxicities classically associated to cancer therapy.

If the subject is identified, using a method according to the present invention, as resistant to a particular treatment of cancer, the method advantageously further comprises a step of selecting a distinct cancer treatment, for example a distinct treatment typically involving a “compensatory molecule” to be used alone or in combination with the originally preselected therapeutic drug(s) or with (a) distinct therapeutic drug(s), as the appropriate therapeutic treatment of cancer for the subject.

Inventors herein identify predictive biomarkers that are able to secure identification of cancer patients prone to respond or resist to a proposed therapy, preferably to a therapy combining i) an immunotherapeutic agent, and ii) an anti-angiogenic agent. These biomarkers are preferably used in a method of predicting, assessing or monitoring the sensitivity or resistance of a subject having a cancer to such a therapy after one or several anti-cancer treatment steps in the subject.

The immunotherapeutic agent is typically a protein such as an antibody, preferably a monoclonal antibody, a cytokine, or a nucleic acid sequence, typically a ribonucleic acid (RNA) or desoxyribonucleic acid (DNA), encoding for such immunotherapeutic agent (and preferably delivered into patients via a viral or chemical vector).

In a particular aspect, the immunotherapy is an immunotherapy combining at least two immunotherapeutic agents, for example two monoclonal antibodies such as in particular an anti-PD-1 monoclonal antibody or an anti-PD-L1 monoclonal antibody and an anti-CTLA4 antibody. In a preferred aspect, the immunotherapeutic agent is an anti-PD-1, or anti-PD-L1, monoclonal antibody.

Preferably, the anti-PD-1 monoclonal antibody(ies) is/are selected from pembrolizumab, also known as lambrolizumab (MK-3475, Merck/MSD), nivolumab (BMS-936558, MDX-1106 or ONO-4538, Bristol-Myers Squib), cemiplimab (Regeneron/Sanofi), dostarlimab (Glaxo Smith Kline), balstilimab (AGEN2034, Agenus), tislelizumab (BGB-A317, Beigene/Novartis), and any combination thereof. In a particular aspect, the anti-PD-1 monoclonal antibody(ies) is/are selected from pembrolizumab, nivolumab, cemiplimab and dostarlimab. Preferred examples are pembrolizumab and nivolumab. A particularly preferred example is pembrolizumab, in particular an intra-venous (I.V.) pembrolizumab formulation.

Relevant examples of anti-PD-L1 monoclonal antibodies are atezolizumab (MPDL3280A, Roche/Genentech), durvalumab (MEDI4736, MedImmune LLC), and avelumab (MSB0010718C, Pfizer/Merck Serono). A preferred example is atezolizumab.

Cytokines can be selected for example from interferon alpha 2a and alpha 2b, IL-2 (proleukin) or their derivatives and cytokine/mAb complexes (also termed antibody drug conjugates or ADC, consisting for instance of a cytokine such as IL-2 associated to an anti-CTLA4 mAb). The cytokine is preferably selected from IFNα2a (ROF) and IL-2.

The anti-angiogenic agent is for example an anti-VEGF agent such as for example bevacizumab (Roche), an anti-VEGFR agent such as for example ramucirumab (Eli Lilly) and/or a tyrosine kinase inhibitor (TKI) such as for example nintedanib (Boehringer Ingelheim). A particularly preferred example is nintedanib, in particular a nintedanib oral formulation.

Inventors reveal for the first time that the presence, preferably the proportion, of a particular subset of CD8+ T cells among live cells CD3+CD45+ T cells (also herein identified as “CD8+CD3+CD45+ T cells”) from a tumor sample of a subject can be used as an efficient biomarker to predict response to a combination therapy involving i) an immunotherapeutic agent and ii) an anti-angiogenic agent. They also discovered that CD4+ T cells expressing the alpha-4 beta-7 integrin (a4b7+ EM CD4+ T cells), cutaneous lymphocyte antigen selectin (CLA+ EM CD4+ T cells), CD49a integrin (CD49a+ EM CD4+ T cells) and/or CXCR3 chemokine receptor (CXCR3+ Th1 CD4+ T cells), VEGFA protein, VEGFD protein, CXCL8 protein, IL6 protein, and/or the number of somatic alterations [or a somatic copy number alteration score or genomic instability score (“SCNA score”)] in cancerous cells of a tumor sample, can be used indifferently as well, independently or in combination, as efficient biomarkers for the same purpose.

In the context of the present invention, the patient or subject is a mammal. In a particular embodiment, the mammal is a human being, whatever its age or sex. The patient typically has a tumor. Unless otherwise specified in the present disclosure, the tumor is a cancerous or malignant tumor, in particular a carcinoma, a melanoma, a lymphoma, a blastoma, or a sarcoma.

In the present invention, the cancer is a cancer that is usually or conventionally treated with one of the herein above described immunotherapy, preferably with an anti-PD-1 or anti-PD-L1 monoclonal antibody, with an anti-angiogenic agent, or with a combination thereof.

The cancer or tumor is typically selected from melanoma, lung, in particular non-small cell lung cancer (NSCLC) or small cell lung cancer (SCLC), head and neck cancer, in particular Head and Neck Squamous Cell Carcinoma (HNSCC), bladder cancer, in particular Urothelial Carcinoma (UC), mesothelioma (“Malignant Mesothelioma” or “MM”), oesophagus cancer, stomach cancer, hepatocarcinoma cancer, kidney or renal cancer, breast cancer, in particular triple negative breast cancer, Epithelial Ovarian Cancer (EOC), and more generally any cancer amenable to immune checkpoint blockade or leading to stimulation of the immune system.

In a particular and preferred aspect, the cancer is a mesothelioma, for example a sarcomatoid mesothelioma or an epithelioid mesothelioma. Mesothelioma is a type of cancer that develops from the thin layer of tissue that covers many of the internal organs (known as the mesothelium) and which is caused by asbestos. This cancer can affect the lungs, heart, abdomen or testes. In the context of the present invention, the mesothelioma is in particular an advanced mesothelioma. In a preferred aspect, the mesothelioma is a lung (i.e., pleural) mesothelioma or a peritoneal mesothelioma.

In the context of mesothelioma, a particular subpopulation of subjects is composed of subjects suffering from advanced mesothelioma, for example locally advanced mesothelioma, possibly a subpopulation of subjects having undergone at least partial tumor resection, suffering of non-operable mesothelioma, and/or having metastasis. The subpopulation of subjects may also suffer of adverse events (“AE”) of grades 1 to 3 (cf. Table 3 of the experimental part).

In a particular aspect of the present invention, the method of the invention is performed after at least partial, for example total, resection of the cancerous tumor and/or metastases thereof, in the subject. The method can however also be performed on the subject before any surgical step.

Preferably the subject is a subject who has not been previously exposed to a treatment of cancer (but may have been exposed to tumor ablation via surgery), or a subject who has received a chemotherapeutic drug (for example a platinum-based chemotherapy, or the combination of a chemotherapeutic agent and an anti-VEGF agent such as bevacizumab) but who has not been treated with (i.e., who has never been exposed to) immunotherapy (i.e., who is naïve to immunotherapy) in particular with an immunotherapeutic treatment involving an anti-PD-1 or anti-PD-L1 agent, in particular anti-PD-1 or anti-PD-L1 monoclonal antibody.

Preferably, the step of determining the presence, absence or expression level of at least one biomarker, for example at least two, three or four biomarkers, in a biological sample of the subject is performed after one or several (for example two, three or more than three) cancer therapeutic steps (i.e., lines of treatment), preferably chemotherapeutic treatment steps.

The herein described new biomarkers allow to select for the therapeutic option involving the combination of an immunotherapeutic agent, in particular an anti-PD-1, or anti-PD-L1, monoclonal antibody, and ii) an anti-angiogenic agent, only the patients who could benefit from such a combination. Therefore, the present invention advantageously allows for the first time to:
prescribe those treatments only to patients who will benefit from them;
avoid to expose patients to severe adverse events if they have no chances of disease response to these treatments;
avoid costly prescriptions in 45% of patients who will not benefit from the treatment;
stratify patient care and help physicians and companies to design new clinical trials and treatment strategies for patients who either express or do not express, preferably in particular proportions, any of these biomarkers;
stratify patients at risk in randomized trials; and
identify a new patient population with specific needs of new treatments.

The method typically comprises a step a) of determining, in a biological sample from said subject, preferably a sample comprising immune cells, stromal cells and/or tumor cells, of the subject, the presence, absence or expression level of at least one biomarker, for example at least two, three, four or more biomarkers selected from the herein described biomarkers, and, when the expression level is determined, a step b) of comparing said expression level to reference expression level(s) or to reference expression ratio(s), thereby predicting, assessing or monitoring whether the subject having a cancer is responsive or resistant to the proposed combination therapy.

The tumor sample is preferably a fresh tumor sample.

In a particular aspect, the tumor sample is obtained with a needle selected from a 16G to 20G needle for tumor biopsy, typically a 18G needle, before being used in a method as herein described.

In a preferred aspect, the fresh tumor sample is being put immediately in a volume between 200 μL and 1 mL, for example 250 or 500 μl, of a preservation or physiological medium, such as for example physiological serum (saline or glucose) or Roswell Park Memorial Institute (RPMI) 1640 Medium, or Dulbecco's Modified Eagle Medium (DMEM), before any step a) as herein described, and/or before any collection of cancerous cells from a tumor sample, or analyses of the tumor biopsy's secretome, in a method as herein described.

In a preferred aspect, the fresh tumor sample is dissociated with both enzymatic and mechanical procedures before being stained and used in a method as herein described.

Examples of enzymatic procedures involve the use of putting tumor samples for digestion for one hour into a gentle MACS OctoDissociator 158 (Miltenyi Biotec) in a dissociation medium such as RPMI 1640 (GIBCO, 31870-025) containing the following enzymes Collagenase IV at 159 50 IU/mL (Sigma-Aldrich, C2139), Hyaluronidase at 280 IU/mL (Sigma-Aldrich, H6254), and 160 DNAse I at 30 IU/mL (Sigma-Aldrich, 260913)). Methods of enzymatic dissociation are described for example in the following reference Dubuisson, A., Fahrner, J.-E., Goubet, A.-G., Terrisse, S., Voisin, N., Bayard, C., Lofek, S., Drubay, D., Bredel, D., Mouraud, S., et al. (2021). Immunodynamics of explanted human tumors for immuno-oncology. EMBO Mol. Med. 13, e12850.

Mechanical procedures usable in the context of the present invention consist in dissociating the tumor sample with the top of a 2-mL syringe plunger in a wet 70-μm filter placed at the top of a 50-mL centrifuge tube. Methods of mechanical dissociation are described for example in the following references: Danlos, F.-X., Texier, M., Job, B., Mouraud, S., Cassard, L., Baldini, C., Varga, A., Yurchenko, A. A., Rabeau, A., Champiat, S., et al. (2023). Genomic Instability and Protumoral Inflammation Are Associated with Primary Resistance to Anti-PD-1+Antiangiogenesis in Malignant Pleural Mesothelioma. Cancer Discov. 13, 858-879.

Another preferred tumor sample is a supernatant sample of a fresh tumor sample, preferably of a fresh tumor sample placed in a preservation medium, for example physiological serum, immediately after the biopsy. In a particular preferred aspect, the supernatant sample (also herein identified as “culture media secretome”) is taken and analyzed (i.e., profiled) after a at least one minute incubation step of the tumor biopsy, for example after an incubation step in a medium (for example physiological medium) of between 1, 2 or 3 minutes and 40 minutes, preferably at least 30 minutes, and up to 72 hours, for example 24 hours.

In another particular aspect, the profiled secretome is a Tumor Interstitial Fluid (TIF) or “tumor juice” secretome.

In another particular aspect, the profiled secretome is a Tumor Proximal Fluid (TPF) derived secretome. In such a context, the analyse or profiling of the secretome may be performed directly in vivo.

In another particular aspect, the biological sample is a blood sample, preferably a fresh whole blood sample, or a part thereof such as plasma or serum.

Blood is typically collected in a tube comprising an anticoagulant agent. Then, blood cells may be separated from plasma by double ultracentrifugation for subsequent analyses thereof.

In a first aspect, inventors herein describe an in vivo, in vitro or ex vivo method of predicting, assessing or monitoring the sensitivity or resistance of a subject having a cancer to a therapy combining i) an immunotherapeutic agent, in particular an anti-PD-1, or anti-PD-L1, monoclonal antibody, and ii) an anti-angiogenic agent, preferably after one or several treatment steps in the subject. The method typically comprise a step a) of determining in a sample of the subject, preferably a sample comprising immune cells and/or tumor cells, typically a tumor sample, of the subject, the presence, preferably the proportion of CD8+ T-cells among live CD45+CD3+ T cells, and a step b) of comparing said live CD8+CD3+CD45+ T cells proportion to a CD8+CD3+CD45+ T cells reference proportion, a proportion of live CD8+CD3+CD45+ T cells below (<) the CD8+CD3+CD45+ T cells reference proportion being indicative of resistance of the subject to the combination therapy and a proportion of live CD8+CD3+CD45+ T cells superior or equal to (≥) the CD8+CD3+CD45+ T cells reference proportion being indicative of sensitivity of the subject to the combination therapy.

As explained herein above, the proportion of CD8+ T cells among live CD45+CD3+ T cells reference is preferably the proportion of CD8+ T cells among live CD45+CD3+ T cells in the tumor of the subject before any exposition of the subject to immunotherapy (i.e., before any immunotherapeutic treatment step in the subject).

In a preferred aspect, CD45+CD3+CD8+ T cells being typically between 0% and 80% of tumor infiltrative CD45+CD3+ T-cells, the threshold value impacting the outcome (“CD8+CD3+CD45+ T cells reference proportion”) is between 50% and 60%, preferably at 55%.

The in vivo, in vitro or ex vivo method of predicting, assessing or monitoring the sensitivity or resistance of a subject having a cancer to a therapy combining i) an immunotherapeutic agent, in particular an anti-PD-1, or anti-PD-L1, monoclonal antibody, and ii) an anti-angiogenic agent, preferably after one or several treatment steps in the subject may otherwise comprise a step a) of determining in a sample of the subject, preferably in a blood sample of the subject, the expression level of EM CD4+ T cells expressing the alpha-4 beta-7 integrin (a4b7+ EM CD4+ T cells), cutaneous lymphocyte antigen selectin (CLA+ EM CD4+ T cells), CD49a integrin (CD49a+ EM CD4+ T cells) and/or CXCR3 chemokine receptor (CXCR3+ Th1 CD4+ T cells), and a step b) of comparing said expression level(s) to a4b7+ EM CD4+ T cells, CLA+EM CD4+ T cells, CD49a+ EM CD4+ T cells and/or CXCR3+ Th1 CD4+ T cells reference expression level(s), (an) expression level(s) superior or equal to (≥) the reference expression level(s) being indicative of sensitivity of the subject to the combination therapy, and (an) expression level(s) below (<) the reference expression level(s) being indicative of resistance of the subject to the combination therapy.

If the sample is blood sample, said blood sample may be a whole blood sample, a plasma sample or a serum sample. It is preferably a plasma sample. It is also preferably a fresh blood sample.

In a preferred aspect, the a4b7+ EM CD4+ T cells, CLA+ EM CD4+ T cells, CD49a+ EM CD4+ T cells and/or CXCR3+ Th1 CD4+ T cells reference proportion level(s) are respectively chosen:
for a4b7+ EM CD4+ T cells, which are typically between 0% and 4% of EM CD4+ T-cells, the threshold value impacting the outcome is in a particular aspect between 0.8% and 1.2%, preferably at 1%;
for CLA+ EM CD4+ T cells, which are typically between 0% and 6% of EM CD4+ T-cells, the threshold value impacting the outcome is in a particular aspect between 1.8% and 2%, preferably at 1.9%;
for CD49a+ EM CD4+ T cells which are typically between 0% and 8% of EM CD4+ T-cells, the threshold value impacting the outcome is in a particular aspect between 2.4% and 2.6%, preferably at 2.5%;
for CXCR3+ Th1 CD4+ T cells which are typically between 0% and 10% of EM CD4+ T-cells, the threshold value impacting the outcome is in a particular aspect between 2.6% and 3.2%, preferably at 3%.

In a particular aspect, the method to analyse the phenotype of immune, stromal and/or cancer cells from the fresh tumor sample or fresh whole blood sample is based on conventional and well-known methods, for example on conventional or spectral flow cytometry (cf. Pitoiset, F., Cassard, L., El Soufi, K., Boselli, L., Grivel, J., Roux, A., Klatzmann, D., Chaput, N., and Rosenzwajg, M. (2018). Deep phenotyping of immune cell populations by optimized and standardized flow cytometry analyses. Cytometry. A 93, 793-802).

The in vivo, in vitro or ex vivo method of predicting, assessing or monitoring the sensitivity or resistance of a subject having a cancer to a therapy combining i) an immunotherapeutic agent, in particular an anti-PD-1, or anti-PD-L1, monoclonal antibody, and ii) an anti-angiogenic agent, preferably after one or several treatment steps in the subject may otherwise comprise a step a) of determining in a sample of the subject, preferably in a blood plasma sample or in a tumor supernatant sample of the subject, the concentration of VEGFA, VEGFD, CXCL8 (IL8) and/or IL6 protein(s), and a step b) of comparing said concentration(s) to VEGFA, VEGFD, CXCL8 and/or IL6 protein(s) reference concentration(s), concentration(s) superior or equal to (≥) the reference concentration(s) being indicative of resistance of the subject to the combination therapy, and concentration(s) below (<) the reference concentration(s) being indicative of sensitivity of the subject to the combination therapy.

Thus, the method of the invention may comprise a step of dosing, for example via ELISA, at least one (bio)marker selected from VEGF, in particular VEGFA and/or VEGFD, IL8 (CXCL8) and/or IL6, in a (preferably fresh) blood sample (for example blood plasma sample) or in the supernatant (also herein identified as “secretome”) of a (preferably fresh) tumor sample biopsy after an incubation step of said tumor sample of at least one minute, for example after an incubation step of between 1, 2, 3, 30, 40 or 45 minutes, preferably at least 30 minutes.

In a preferred aspect, VEGFA is dosed in the secretome of a (preferably fresh) tumor sample biopsy and/or VEGFD is dosed in a blood sample, both samples being obtained from the subject to be tested.

In another particular aspect, the method comprises a step of dosing at least one marker selected from VEGFA, IL6, IL8 (CXCL8) and granzyme (GZMA and/or GZMB) in the supernatant of the tumor sample after an incubation step of said tumor sample of at least 1 minute, for example 2, 3, 10, 30, 40 or 45 minutes, or 24 h and up to 72 h, or a step of dosing at least one of said markers in the plasma obtained after double centrifugation of the blood sample, preferably of the fresh whole blood sample.

Dosing of said markers may be performed via any well-known method, such as for example ultrasensitive titration assay, Luminex, Meso Scale Discovery (MSD), etc.

In a particular aspect, the reference concentration of VEGFA in the secretome being typically between 0 and 20000 pg/mL, the threshold value impacting the outcome is between 500 and 700 pg/mL, preferably at 600 pg/mL. In a particular aspect, the reference concentration of VEGFD in the plasma being typically between 1 and 6 pg/mL, the threshold value impacting the outcome is between 3 and 4 pg/mL, preferably at 3.5 pg/mL. In a particular aspect, the reference concentration of IL8 (CXCL8) in the plasma being typically between 2 and 25 pg/mL, the threshold value impacting the outcome is between 8 and 12 pg/mL, preferably at 10 pg/mL.

In a particular aspect, the reference concentration of IL6 in the plasma being typically between 5 and 55 pg/mL, the threshold value impacting the outcome is between 12 and 18 pg/mL, preferably at 15 pg/mL.

In another particular aspect, the reference concentration is the concentration of the marker at baseline, i.e. before administration to the subject of an immunotherapeutic agent, in particular an anti-PD-1, or anti-PD-L1, monoclonal antibody such as pembrolizumab, for example of a combination of i) an immunotherapeutic agent, in particular an anti-PD-1, or anti-PD-L1, monoclonal antibody such as pembrolizumab, and ii) an anti-angiogenic agent such as nintedanib.

The in vivo, in vitro or ex vivo method of predicting, assessing or monitoring the sensitivity or resistance of a subject having a cancer to a therapy combining i) an immunotherapeutic agent, in particular an anti-PD-1, or anti-PD-L1, monoclonal antibody, and ii) an anti-angiogenic agent, preferably after one or several treatment steps in the subject may otherwise comprise a step of determining in cancerous cells of a tumor sample of the subject, the number of somatic alterations, a somatic copy number alteration score or genomic instability score (“SCNA score”) above (>) a reference score being indicative of resistance of the subject to the combination therapy, and a SCNA score equal to or below (≤) the reference score being indicative of sensitivity of the subject to the combination therapy.

The somatic copy number alteration score (“SCNA score” or “SCNA genomic instability score”) may be established as follow: for each tumor sample, the absolute gene copy number (ACN) profile is generated by a method to derive copy number profiles of tumor cells such as ASCAT (Allele-Specific Copy Number Analysis of Tumors) [cf. Van Loo P et al., PNAS 2010 Allele-specific multi-sample copy number segmentation. Ross E M, Haase K, Van Lo P & Markowetz F. Bioinformatics (2020)] through the EaCoN (Easy Copy Number) algorithm. For each chromosome taken independently, the basal ACN level is identified as the one with the longest total width. Then, for each chromosome, ACN levels are converted into the absolute difference in copy number (aDCN) to this basal level (i.e., if basis was 3 copies, a 1 copy segment value will be 2; 2 copies=>1; 3 copies=>0; 4 copies=>1; etc.). The final SCNA score is computed as the width-ponderated sum of each of these converted CN-to-basis values, divided by the total covered genome length.

Implementations of the methods of the invention involve obtaining a (biological) sample from a subject. The sample can be a fluid sample and may include any specimen containing immune cells such as blood, lymphatic fluid, spinal fluid, pleural effusion, ascites, or a combination thereof. The biological sample is preferably a sample comprising tumor cells. Such a sample can be a tumor biopsy, a whole tumor piece, a tumor bed sample, a metastatic lymph node cells sample, or a combination thereof. The sample is preferably a fresh tumor sample biopsy or a tumor sample biopsy which has not been frozen. In a particular aspect, the fresh tumor sample biopsy or tumor sample biopsy which has not been frozen, is dissociated with both enzymatic and mechanical procedures before being stained and used in the context of a herein described method or use.

The determination of the SCNA score may be performed on a fresh tumor sample or on a formalin-fixed or frozen tumor tissue.

Several or each of the herein above described possible steps (“a)” in particular) may be performed in the context of the in vivo, in vitro or ex vivo method of the invention of predicting, assessing or monitoring the sensitivity or resistance of a subject having a cancer to a therapy combining i) an immunotherapeutic agent and ii) an anti-angiogenic agent, preferably after one or several (anti-cancer) treatment steps/lines in the subject.

Typically, the expressions “reference value” or “reference expression level” used in the present description is the level, concentration or proportion of the biomarker in a control sample derived from one or more reference subjects (reference population) having a cancer. The reference value or reference expression level is typically the median value obtained from the reference population. The reference value typically varies in a range of values defined for a given population. The reference value can further be a score, a ratio involving two distinct biomarkers or a % or proportion of one several biomarkers in a control sample.

In a particular and preferred aspect, the (bio)marker reference expression level is the level, concentration or proportion of the marker in the tumor of the subject before any immunotherapeutic treatment step in the subject.

Typically, a cell surface biomarker expression can easily be determined by flow cytometry and a molecule release can easily be determined by ultra-sensitive titration assays (e.g., conventional ELISA, Luminex technology or Meso Scale Discovery technology) as indicated herein above, preferably in the context of the method herein described for the first time by inventors wherein the step of dosing at least one marker is performed in the supernatant of a fresh tumor sample biopsy after an incubation step of at least one minute, for example between 1 and 40 minutes, preferably at least 30 minutes, and as further explained below.

In some embodiments of the invention, identification of a biomarker of interest involves use of at least one binding agent. Furthermore, it is contemplated that a binding agent may be specific or not to the considered biomarker. For example, the CD8⁺CD3+CD45+ T cells binding agent may bind to a part of CD8 (e.g. an epitope) that is not available depending on whether it is expressed by/bound to CD45⁺CD3⁺ T cells from a biological sample comprising immune cells as previously described. Alternatively, different conformations may serve the basis for binding agents capable of distinguishing between similar biomarkers.

The binding agent is typically a polypeptide. The polypeptide is, in particular embodiments, an antibody. In further embodiments, the antibody is a monoclonal antibody. The antibody can be bi-specific, recognizing two different epitopes. The antibody, in some embodiments, immunologically binds to more than one epitope from the same biomarker. In some embodiments of the invention, the binding agent is an aptamer.

In some embodiments of the invention, the binding agent is labeled. In further embodiments, the label is radioactive, fluorescent, chemiluminescent, an enzyme, or a ligand. It is also specifically contemplated that a binding agent is unlabeled, but may be used in conjunction with a detection agent that is labeled. A detection agent is a compound that allows for the detection or isolation of itself so as to allow detection of another compound that binds, directly or indirectly. An indirect binding refers to binding among compounds that do not bind each other directly but associate or are in a complex with each other because they bind the same compounds or compounds that bind each other.

When CD45 is to be detected, the antibody to be used can be selected for example clone HI30, (ref 612891, BD Biosciences).

When CD3 is to be detected, the antibody clone to be used can be for example clone UCHT1 (e.g ref 563546, BD Biosciences).

When CD8 is to be detected, the antibody clone to be used can be for example clone SK1 (e.g., ref 560179, BD Biosciences).

When a4b7 is to be detected, the antibody clone to be used can be for example clone Hu117 (e.g., ref MAB10078, R&D systems).

When CLA is to be detected, the antibody clone to be used can be for example Clone HECA-452 (e.g ref 321305, Biolegend).

When CD49 is to be detected, the antibody clone to be used can be for example clone 9F10 (ref 304343, Biolegend).

When CXCR3 is to be detected, the antibody clone to be used can be for example clone 1C6 (ref 567038, BD Biosciences).

When CD4 is to be detected, the antibody clone to be used can be for example SK3 (e.g ref: 3457703, BD Biosciences).

Other embodiments of the invention involve a second binding agent in addition to a first binding agent. The second binding agent may be any of the entities discussed above with respect to the first binding agent, such as an antibody. It is contemplated that a second antibody may bind to the same of different epitopes as the first antibody. It is also contemplated that the second antibody may bind the first antibody or another epitope than the one recognized by the first antibody.

As discussed earlier, binding agents may be labeled or unlabeled. Any polypeptide binding agent used in methods of the invention may be recognized using at least one detection agent. A detection agent may be an antibody that binds to a polypeptide binding agent, such as an antibody. The detection agent antibody, in some embodiments, binds to the Fc-region of a binding agent antibody. In further embodiments, the detection agent is biotinylated, which is incubated, in additional embodiments, with a second detection agent comprising streptavidin and a label. It is contemplated that the label may be radioactive, fluorescent, chemiluminescent, an enzyme, or a ligand. In some cases, the label is an enzyme, such as horseradish peroxidase.

In a particular aspect the methods of the invention involve the use of flow cytometry or ELISA assay to detect the herein described biomarkers.

In some embodiments, the selected flow cytometry technology is FACS (Fluorescence-activated cell sorting). FACS can be used for distinguishing and separating into two or more containers specific cells from a heterogeneous mixture of biological cells, based upon the specific light scattering and fluorescent characteristics of each cell.

In other embodiments, the ELISA assay is a sandwich assay. In a sandwich assay, more than one antibody will be employed. Typically, ELISA method can be used, wherein the wells of a microtiter plate are coated with a set of antibodies which recognize the protein of interest. A sample containing or suspected of containing the protein of interest is then added to the coated wells. After a period of incubation sufficient to allow the formation of antibody-antigen complexes, the plate(s) can be washed to remove unbound moieties and a detectably labelled secondary binding molecule added. The secondary binding molecule is allowed to react with any captured sample marker protein, the plate washed and the presence of the secondary binding molecule detected using methods well known in the art.

In the methods herein described of predicting, assessing or monitoring the sensitivity of a subject having a cancer to an anti-cancer therapy, in particular to a therapy combining i) an immunotherapeutic agent, in particular an anti-PD-1 or anti-PD-L1, monoclonal antibody, and ii) an anti-angiogenic agent, as well as in the methods herein described of selecting an appropriate therapeutic treatment, any classical method known by the skilled person of determining the presence or measuring the expression level of a compound of interest, such as typically FACS, ELISA and radioimmunoassay can be used.

A method of selecting an appropriate, preferably optimal, therapeutic treatment of cancer for a subject having a cancer as herein described, is in addition herein described, as well as appropriate therapeutic treatment involving for example compensatory molecules for use in such a treatment of cancer, possibly in combination with the preselected therapeutic drug(s), typically immunotherapeutic and anti-angiogenic drugs, in a subject identified, using a method as herein described, as resistant to said preselected therapeutic drug(s).

Also herein described is thus a method of selecting an appropriate therapeutic treatment for a subject having a cancer, which method comprises a step of predicting or assessing the sensitivity or resistance of a subject having a cancer or a malignant tumor to a proposed anti-cancer therapy, preferably a treatment combining i) an immunotherapeutic agent, in particular an anti-PD-1, or anti-PD-L1, monoclonal antibody and an anti-angiogenic agent, using a method according to the present invention as described herein above.

If the subject is identified as sensitive to the proposed combination therapy, this means that said therapy is an appropriate therapeutic treatment for the subject.

If the subject is identified as resistant to the proposed combination therapy, this means that said therapy will not be efficient in the subject and will in addition possibly generate unwanted deleterious side effects in the subject. In such circumstances, the method further advantageously comprises an additional step of selecting a distinct or complementary therapeutic treatment of cancer more appropriate for the subject.

For subject suffering of a mesothelioma, when the combination of an anti-PD-1, or anti-PD-L1, monoclonal antibody and of an anti-angiogenic agent is not efficient, or not efficient alone, in the subject, the distinct therapeutic treatment can be a compound selected from any other immunostimulatory monoclonal antibody such as an antibody targeting TIM3, LAG3, VISTA, BTLA, CD137, OX40, ICOS, B7-H3, B7-H4, KIR, IDO, or TIGIT, and any combination thereof, or a combination of the anti-PD-1, or anti-PD-L1, monoclonal antibody and of the anti-angiogenic agent and of such a distinct compound.

Methods of screening for candidate therapeutic agents for preventing or treating cancer are also included as part of the invention. The method is typically a method which is performed in vivo, in vitro or ex vivo. When performed ex vivo, it can be performed for example on a sample from a subject who has been administered with a test compound.

A method herein described is typically a method for screening or identifying a compound suitable for improving the treatment of a cancer in a subject having a cancer, said method comprising determining the ability of a test compound to modify the expression of at least one of the herein described biomarkers of response or resistance to the therapy of interest involving the combination of an anti-PD-1, or anti-PD-L1, monoclonal antibody and of an anti-angiogenic agent, or compensate an abnormal expression thereof.

Further herein described is method of selecting or disqualifying a subject having a cancer for inclusion in a clinical trial, the clinical trial being for evaluating an anti-cancer treatment, preferably a cancer treatment combining an immunotherapeutic agent, in particular an anti-PD-1, or anti-PD-L1, monoclonal antibody and an anti-angiogenic agent, which method comprises a step of predicting or assessing the sensitivity of a subject having a cancer to the combination therapy using a method according to the present invention as described herein above.

The present invention also includes kits for predicting, assessing or monitoring the sensitivity of a subject having a cancer or a malignant tumor to a cancer therapy, in particular a therapy involving the combination of an anti-PD-1, or anti-PD-L1, monoclonal antibody and of an anti-angiogenic agent, wherein the kit comprises, as detection means, possibly in suitable container means, at least two agents, for example three, four or five agents, each of said agent specifically recognizing one of the herein described biomarkers. These at least two agents are typically at least two distinct antibodies, used as detection means, selected from the group consisting of an antibody recognizing a viability dye, an antibody specific to CD45, CD8, CD3, a4b7, CLA, CD49a, CXCR3, CD45RO or CD45RA, CCR7, IL6, IL8 (CXCL8), VEGFA, VEGFD, Granzyme A, or Granzyme B, and, optionally, a leaflet providing the reference expression levels corresponding to anyone of, several or each of said proteins.

In further embodiments, the binding agent is labeled or a detection agent is included in the kit. It is contemplated that the kit may include one, at least one or several, biomarker binding agents attached to a non-reacting solid support, such as a tissue culture dish or a plate with multiple wells. It is further contemplated that such a kit includes one or several detectable agents in certain embodiments of the invention. In some embodiments, the invention concerns kits for carrying out a method of the invention comprising, in suitable container means: (a) agent(s) that specifically recognizes all or part of a given biomarker; and, (b) at least one positive control, for example at least two positive controls, that can be used to determine whether the agent is capable of specifically recognizing all or part of said given biomarker. The kit may also include other reagents that allow visualization or other detection of anyone of the herein described biomarkers, such as reagents for colorimetric or enzymatic assays.

Also herein described is the use of such a kit for predicting, assessing or monitoring the sensitivity of a subject having a tumor to a cancer treatment, in particular a treatment combining an anti-PD-1, or anti-PD-L1, monoclonal antibody and an anti-angiogenic agent, wherein the kit comprises at least two distinct antibodies selected from an antibody recognizing a viability dye, CD45, CD8, CD3, a4b7, CLA, CD49a, CXCR3, CD45RO or CD45RA, CCR7, IL6, IL8 (CXCL8), VEGFA, VEGFD, Granzyme A, and Granzyme B, as detection means, and, optionally, a leaflet providing corresponding reference expression levels.

This disclosure also provides the following, non-limiting embodiments:
1. An in vitro or ex vivo method of predicting, assessing or monitoring the sensitivity or resistance of a subject having a cancer to a therapy combining i) an immunotherapeutic agent, in particular an anti-PD-1, or anti-PD-L1, monoclonal antibody, and ii) an anti-angiogenic agent, after one or several treatment steps in the subject, wherein the method comprises:
a step a) of determining in a tumor sample of the subject, the proportion of CD8+ T-cells among live CD45+CD3+ T cells, and a step b) of comparing said live CD8+CD3+CD45+ T cells proportion to a CD8+CD3+CD45+ T cells reference proportion, a proportion of live CD8+CD3+CD45+ T cells below (<) the CD8+CD3+CD45+ T cells reference proportion being indicative of resistance of the subject to the combination therapy and a proportion of live CD8+CD3+CD45+ T cells superior or equal to (≥) the CD8+CD3+CD45+ T cells reference proportion being indicative of sensitivity of the subject to the combination therapy;
a step a) of determining in a blood sample of the subject, the expression level of effector memory (EM) CD4+ T cells expressing the alpha-4 beta-7 integrin (a4b7+ EM CD4+ T cells), cutaneous lymphocyte antigen selectin (CLA+ EM CD4+ T cells), CD49a integrin (CD49a+ EM CD4+ T cells) and/or CXCR3 chemokine receptor (CXCR3+ Th1 CD4+ T cells), and a step b) of comparing said expression level(s) to a4b7+ EM CD4+ T cells, CLA+ EM CD4+ T cells, CD49a+ EM CD4+ T cells and/or CXCR3+ Th1 CD4+ T cells reference expression level(s), (an) expression level(s) superior or equal to (≥) the reference expression level(s) being indicative of sensitivity of the subject to the combination therapy, and (an) expression level(s) below (<) the reference expression level(s) being indicative of resistance of the subject to the combination therapy;
a step a) of determining in a blood plasma sample or in a tumor supernatant sample of the subject, the concentration of VEGFA, VEGFD, CXCL8 and/or IL6 protein(s), and a step b) of comparing said concentration(s) to VEGFA, VEGFD, CXCL8 and/or IL6 protein(s) reference concentration(s), concentration(s) superior or equal to (≥) the reference concentration(s) being indicative of resistance of the subject to the combination therapy, and concentration(s) below (<) the reference concentration(s) being indicative of sensitivity of the subject to the combination therapy; and/or
a step of determining in cancerous cells of a tumor sample of the subject, the number of somatic alterations, a somatic copy number alteration score or genomic instability score (“SCNA score”) above (>) a reference score being indicative of resistance of the subject to the combination therapy, and a SCNA score equal to or below (≤) the reference score being indicative of sensitivity of the subject to the combination therapy.
2. The method according to embodiment 1, wherein the proportion of CD8+ T cells among live CD45+CD3+ T cells reference is the proportion of CD8+ T cells among live CD45+CD3+ T cells in the tumor of the subject before any immunotherapeutic treatment step in the subject.
3. The method according to embodiment 1 or 2, wherein the anti-PD-1 monoclonal antibody is selected from pembrolizumab, nivolumab, cemiplimab and dostarlimab, or the anti-PD-L1 monoclonal antibody is selected from atezolizumab, durvalumab and avelumab.
4. The method according to any one of embodiments 1 to 3, wherein the anti-angiogenic agent is an anti-VEGF agent, for example bevacizumab, and/or a tyrosine kinase inhibitor (TKI), for example nintedanib.
5. The method according to any one of embodiments 1 to 4, wherein the cancer is a mesothelioma (MM).
6. The method according to any one of embodiments 1 to 5, wherein the tumor sample is a fresh tumor sample or the tumor supernatant sample of a fresh tumor sample.
7. The method according to embodiment 6, wherein the tumor sample is obtained with a needle selected from a 16G to 20G needle for tumor biopsy, typically a 18G needle, before being used in the method according to anyone of embodiments 1 to 6.
8. The method according to embodiment 6 or 7, wherein the fresh tumor sample is being put immediately in a volume between 200 μL and 1 mL of a preservation medium before step a) and/or before any collection of cancerous cells from the tumor sample in the method according to anyone of embodiments 1 to 7.
9. The method according to any one of embodiments 6 to 8, wherein the fresh tumor sample is dissociated with both enzymatic and mechanical procedures before being stained and used in the method according to anyone of embodiments 1 to 8.
10. The method according to any one of embodiments 1 to 5, wherein the blood sample is a fresh whole blood sample.
11. The method according to any one of embodiments 6 to 10, wherein the method comprises a step of dosing via ultrasensitive titration assay at least one marker selected from VEGFA, IL6, IL8 (CXCL8) and granzyme (GZMA and/or GZMB) in the supernatant of the tumor sample after an incubation step of said tumor sample of at least one minute, and up to 72 h, or a step of dosing at least one of said markers in the plasma obtained after double centrifugation of the fresh whole blood sample.
12. The method according to embodiment 10 or 11, wherein the method to analyse the phenotype of immune, stromal and/or cancer cells from the fresh tumor sample or fresh whole blood sample is based on conventional or spectral flow cytometry.
13. A method of selecting an appropriate therapeutic treatment for a subject having a cancer, which method comprises a step of predicting or assessing the sensitivity of a subject having a cancer to a cancer treatment combining an immunotherapeutic agent and an anti-angiogenic agent using a method according to any one of embodiments 1 to 12.
14. A method of selecting or disqualifying a subject having a cancer for inclusion in a clinical trial, the clinical trial being for evaluating a cancer treatment combining an immunotherapeutic agent and an anti-angiogenic agent, which method comprises a step of predicting or assessing the sensitivity of a subject having a cancer to the combination therapy, using a method according to anyone of embodiments 1 to 12.
15. Use of a kit for predicting, assessing or monitoring the sensitivity of a subject having a tumor to a cancer treatment combining an anti-PD-1, or anti-PD-L1, monoclonal antibody and an anti-angiogenic agent, wherein the kit contains at least two distinct antibodies selected from an antibody recognizing a viability dye, CD45, CD8, CD3, a4b7, CLA, CD49a, CXCR3, CD45RO or CD45RA, CCR7, IL6, IL8 (CXCL8), VEGFA, VEGFD, Granzyme A, and Granzyme B, as detection means, and, optionally, a leaflet providing corresponding reference expression levels.

The invention will be further illustrated by the following figures and examples. However, these examples and figures should not be interpreted in any way as limiting the scope of the present invention.

### FIGURES

FIG. 1. Trial design, treatment efficacy and outcome of the PEMBIB Mesothelioma Cohort.

A. Graphical representation of the treatments doses provided and routes of administration (Q3W: intravenous infusion every 3 weeks; bid: oral take twice a day). B. the treatments regimen according to the PEMBIB protocol with the blood and tumor sampling scheme. C. Waterfall plot depicting the Best Objective Response (BORR) on target lesions according to RECIST1.1 criteria. D. Spider plot of sum of target lesions upon treatment. E. Swimmer plot illustrating the individual response and clinical benefit status according to their tumor histology.

Durable Clinical Benefit (DCB) is defined as being in complete or partial response or stable disease at 6 months per RECIST1.1 criteria. F. Kaplan-Meier Survival Curves depicting the overall survival of the patients treated according to their durable clinical benefit status at 6 months. Abbreviations: E=Ethelioid; B=Biphasic; S=Sarcomatoid; DCB=Durable Clinical Bene fit.

FIG. 2. Baseline tumor and systemic immune features of patients with benefit to pembrolizumab and nintedanib.

A. Graphical representation of the techniques applied to fixed, frozen and fresh whole blood and tumor samples prospectively collected and analyzed in the study. B. Membrane PD-L1 expression by cancer cells by immuno-histo-chemistry (anti-PDL1 staining clone SP263; TPS scoring) on baseline biopsies (n==20) according to DCB status. C. Gene Set Enrichment Analysis plot (GSEA; “Hallmark” panel) from RNAseq data representing the genomic pathways significantly upregulated (activated) or downregulated (suppressed) in baseline tumor biopsies of patients with DCB (compared to those without DCB); n=13 tumor samples, considering all genes differentially expressed (p-value≤0.05 adjusted with Benjamin-Hochberg method [Wald's test]; n=515 genes. D. Evaluation of tumor immune infiltrate by flow cytometry after fresh tissue dissociation of baseline tumor biopsies between patients with DCB or No DCB (n==22). E. Analyses of fresh blood samples by flow cytometry to phenotype circulating T lynphocvtes before treatment initiation. Boxplot representation of percentage of circulating CXCR3+ CCR4− CCR6− cells among helper memory CD4+ T cells. F. Boxplot representations of percentages of alpha-4 beta-7 positive (a4b7-r), cutaneous lymphocyte-associated antigen positive (CLA+) and CD49a positive (CD49a+) cells among circulating effector memory (EM) CD4+ and CD8+ T cells (n=25). All tests were Wilcoxon rank-sum test (unpaired samples). Representation of p-value: ns >0.05, * ≤0.05, ** ≤0.01, *** ≤0.001.

FIG. 3. Pharmacodynamic markers of nintedanib monotherapy and upon addition of pembrolizumab.

A. Paired plasmatic levels of Angiopoietin-2, CCL21 and CCL23 before nintedanib lead-in monotherapy treatment (D-7) and after seven days of daily treatment (C1D1). B. Paired plasmatic levels of soluble PD-1, C soluble PD-L1 and D CCL19 after one week of nintedanib and before addition of pembrolizumab (C1D1) and after seven days of combination therapy (C1D8) (n=25). E. Evolution of paired plasma CXCIL9, CXCL10, CXCL11 and CXCL13 concentrations between cycle 1 day 1 (C1D1) and after one week of nintedanib+pembrolizumab combination therapy (C1D8) for patients with and without DCB (n=25). All statistical tests were paired Wilcoxon signed-rank test (paired samples). Representation of p-value: ns >0.05, * 0.05, ** ≤0.01, *** ≤0.001, **** ≤0.0001.

FIG. 4. Patients with No DCB effectively recruit cytotoxic CD8+ T-cells in their tumors upon pembrolizumab+nintedanib therapy.

A. Volcano plot depicting the most secreted chemokines in tumor secretome between baseline and C2D1 tumor biopsies (prior second infusion of pembrolizumab). B. Paired comparisons of CXCL9 concentrations in the secretome of biopsies for patients with DCB or No DCB. C. Paired comparisons of CD45+ immune cells by proportions (percentage among total viable cells; left panel) and counts (absolute cell number; right panel) in tumor biopsies before (screening) and after 3 weeks of combination treatment (C2D1) in patients with DCB or No DCB. D. Paired comparisons of CD3+ T-cells by proportions (percentage among total viable cells; left panel) and counts (absolute cell number; right panel) in tumor biopsies before (screening) and after 3 weeks of combination treatment (C2D1) in patients with DCB or No DCB. E. Paired comparisons of CD8+ T-cells by proportions (percentage among total viable cells; left panel) and counts (absolute cell number; right panel) in tumor biopsies before (screening) and after 3 weeks of combination treatment (C2D1) in patients with DCB or No DCB. F, G, H, I, J. CD8 alpha (CD8A), Eomesodermin (EOMES), T-box transcription factor 21 (TBX21), (iranulysin (GNLY), Granzyme A (GZMA; left) and B (GZMB; right) respective gene expression from RNAseq data (normalized read counts) before (screening) and after 3 weeks of combination treatment (C2D1) in patients with DCB or No DCB. K. Absence of difference in the percentage of CD45+ immune cells among total viable cells (left panel), CD3+ T-cells among CD45+ viable immune cells (middle panel) and CD8+ T-cells among viable CD3+ T-cells (right panel) in tumor biopsies of patients with No DCB compared to patients with DCB. All tests w ere paired Wilcoxon signed rank test (paired comparisons) and Wilcoxon rank-sum test (unpaired comparisons); representation of p-value: ns >0.05, * ≤0.05, ** ≤0.01, * ≤0.001.

FIG. 5. Patients with No DCB actively recruit CD4+ T-cells with regulatory phenotype in their tumors upon pembrolizumab+nintedanib therapy.

A. Paired comparisons of CD4+ T-cells by proportions (percentage among total viable cells; left panel) and counts (absolute cell number; right panel) in tumor biopsies before (screening) and after 3 weeks of combination treatment (C2D1) in patients with DCB or No DCB. B, C. Respective proportions of CD4+ cells among CD3+ T-cells and CD4+CD25+ cells among CD4+ T-cells in tumor biopsies of patients with No DCB compared to patients with DCB after 3 weeks of combination treatment (C2D1). D. Dynamics of CD4+CD25+ T-cells absolute cell counts in tumor biopsies of patients with No DCB compared to patients with DCB before (screening) and after 3 weeks of combination treatment (C2D1). E. Soluble CD25 in the secretome of tumor biopsies of patients with No DCB compared to patients with DCB after 3 weeks of combination treatment (C2D1). F, G, H, I TIGIT, ICOS, CTLA4 and FOXP3 respective gene expression from RNAseq data (normalized read counts) before (screening) and after 3 weeks of combination treatment (C2D1) in patients with DCB or No DCB. All tests were paired Wilcoxon signed rank test (paired comparisons) and Wilcoxon rank-sum test (unpaired comparisons); representation of p-value: ns >0.05, * ≤0.05.

FIG. 6. Primary Resistance to Pembrolizumab+Nintedanib is associated with high VEGF, CXCL8 and IL-6 concentrations in the blood and tumor of mesothelioma patients.

A. Paired comparisons of VEGF-A concentrations in the secretome of tumor biopsies between baseline (screening) and after 3 weeks of combination treatment (C2D1) in patients with DCB and No DCB. B. Comparison of median valies of VEGF-A concentrations in the secretome of tumor biopsies after 3 weeks of combination treatment (C2D1) of patients with DCB and No DCB. C-D. Comparison of median values of VEGF-D concentrations in the plasma at baseline (day-7) of patients with DCB and No DCB (left panel (6C)), and paired dynamics of VEGF-D plasma concentrations during the first week of nintedanib monotherapy (D-7 vs C1D1; right panel (6D)). E. Comparison of median CXCL8 concentration values in the plasma of DCB vs No DCB patients at baseline (day-7). F. Paired comparisons of CXCL8 concentrations in the plasma of DCB vs No DCB patients between baseline (D-7) and after 2 weeks of nintedanib and one week of pembrolizumab (C1D8). G. Paired comparisons of CXCL8 concentrations in the secretome of tumor biopsies of DCB vs No DCB patients between baseline (screening) and after 3 weeks of combination treatment (C2D1). H. Comparison of median values of CXCL8 concentrations in the secretome of tumor biopsies after 3 weeks of combination treatment (C2D1) in patients with DCB and No DCB. I, J, K. Comparisons of median IL-6 concentration values in the plasma of DCB vs No DCB patients at baseline (day-7), after 7 days of nintedanib monotherapy (C1D1) and after 7 days of combination treatment (C1D8) respectively. L. Paired comparisons of IL-6 concentrations in the secretome of tumor biopsies of DCB vs No DCB patients between baseline (screening) and after 3 weeks of combination treatment (C2D1). M. Comparison of median IL-6 concentrations (BioRad assay) in the secretome of tumor biopsies from patients with DCB vs No DCB patients after 3 weeks of combination treatment (C2D1). All tests were Wilcoxon rank-sum test (not paired samples) and paired Wilcoxon signed rank test (paired samples) (representation of p-value: ns >0.05, * ≤0.05).

FIG. 7. Somatic Copy Number Alterations are higher and correlate with IL-6 levels in patients with primary resistance to pembrolizumab+nintedanib.

A. Representative pictures of fluorescence in situ hybridization (FISH) analyses of 9p21 chromosomal region on FFPE embedded tumor biopsy samples from a patient with DCB (Patient #45, C2D1 biopsy, 9p21 heterozygous deletion; left) and No DCB (Patient H16, screening biopsy, chromosome 9 monosomy; right). B. Proportions of Chromosome 9 alterations in patients with DCB (left) and No DCB (right). 2/9 (29%) tumor biopsies presented chromosome 9 monosomy in DCB patients. Patients with No DCB showed 1/11 (9.1%) chromosome 9 monosomy, 2/11 (18%) 9p21 heterozygous deletions, and 4/11 (36%) 9p21 homozygous deletions. C. Oncoplot representation of the principal genomic alterations found by WES analyses on tumor biopsies. Of note, uniparental disomia, is a copy neutral loss of heterozygosity. D. Comparison of the median score of Somatic Copy Number Alterations (SCNA) in tumor biopsies of patients with DCB and No DCB. Wilcoxon rank-sum test (unpaired samples); representation ofp-value: ns >0.05, * ≤0.05. E. Linear correlation between somatic copy number alteration score (SCNA score) on tumor biopsies and IL-6 plasma levels (Siemens assay). F. Unsupervised clustering heatmap of the principal biomarkers identified illustrating the value of data generated on fresh samples to predict the outcomes of patients treated with anti-PD1 and anti-angiogenics in patients with mesothelioma.

FIG. 8. PD-L1 protein expression by immuno-histochemistry scoring according to the RECIST response.

Membrane PD-L1 expression by cancer cells by immune-histo-chemistry (anti-PDL1 staining clone SP263; TPS scoring) on baseline biopsies (n=20) according to RECIST 1.1 status (PD: Progressive Disease; SD: Stable Disease; PR: Partial Response). Volcano plot representation of DGE between baseline tumor biopsies from patients with or without DCB (log-2 fold change ≤−3 or ≥1 with p-value≤0.01 adjusted with Beniamini-Iochberg method [Wald's test]) (n=13).

FIGS. 9A-9B. Further descriptions of tumor immune infiltrates.

A. Besides PD-L1 scoring, Immuno-Histo-Chemistry on baseline tumor biopsies did not find significant differences between patients with DCB and No DCB. Scoring of tumor cells, biopsy surface area, CD8 scoring, CD3+CD8− scoring, FOXP3 scoring, CD20 scoring, tertiary lymphoid structures scoring, macrophages (CD68) scoring, CD68+CD163− (M1) macrophages scoring and CD68+CD163+(M2) macrophages scoring on baseline tumor biopsies of patients with DCB and No DCB. B. Proportions of PD1+CD4+ & PD1+CD8+ T-cells in baseline tumor biopsies by flow cytometry on fresh tumor biopsies according to their outcome.

FIG. 10. Significant dynamic differences upon treatment in circulating chemokines.

A., B. During the first week of nintedanib lead-in monotherapy CCL15 and CXCL10 plasma levels decreased whereas CCL8 increased in patients with No DCB but decreased in patients with DCB. C. CCL3, CCL8 and CCL23 plasma concentrations increased more in patients with No DCB during the first week of combination therapy, whereas CXCL2 decreased.

FIG. 11. Gating strategy for flow cytometry analyses of T cells infiltration on tumor biopsies.

Top: Example of a patient with high immune infiltrates in its tumor biopsy. Bottom: Example of a patient with low immune infiltrates in his tumor.

FIG. 12. Cytotoxic response in tumor biopsies of patients with No DCB upon combination treatment.

A., B. CD3e, CD3z and Interferon Gamma (IFNG) gene expression are significantly upregulated in patient with No DCB between baseline (screening) and at 3 weeks combination therapy (C2D1) respectively. C. Evolution of the tumor infiltration by immune subsets (T-cells, CD8 T-cells, NK Cells) and cytotoxic score between screening and C2D1 in patients with and without DCB, by immune deconvolution of RNASeq (MCP Counter). D. CD8+ scoring by IHC between patients with DCB and No DCB on tumor biopsies at C2D1 (left panel), dynamics between screening and C2D1 (middle left panel), scoring according to RECIST response status on screening biopsies (middle right panel) and at C2D1 (tight panel). E. Proportions of CD25+ cells among CD4+ T-cells in tumor biopsies at screening according to DCB status (left) or RECIST1.1 best objective response (right). F. Concentrations of VEGF-A in the plasma of patients at C1D-7, C1D1 and C1D8.

Throughout this application, various references describe the state of the art to which this invention pertains. The disclosures of these references are hereby incorporated by reference into the present disclosure.

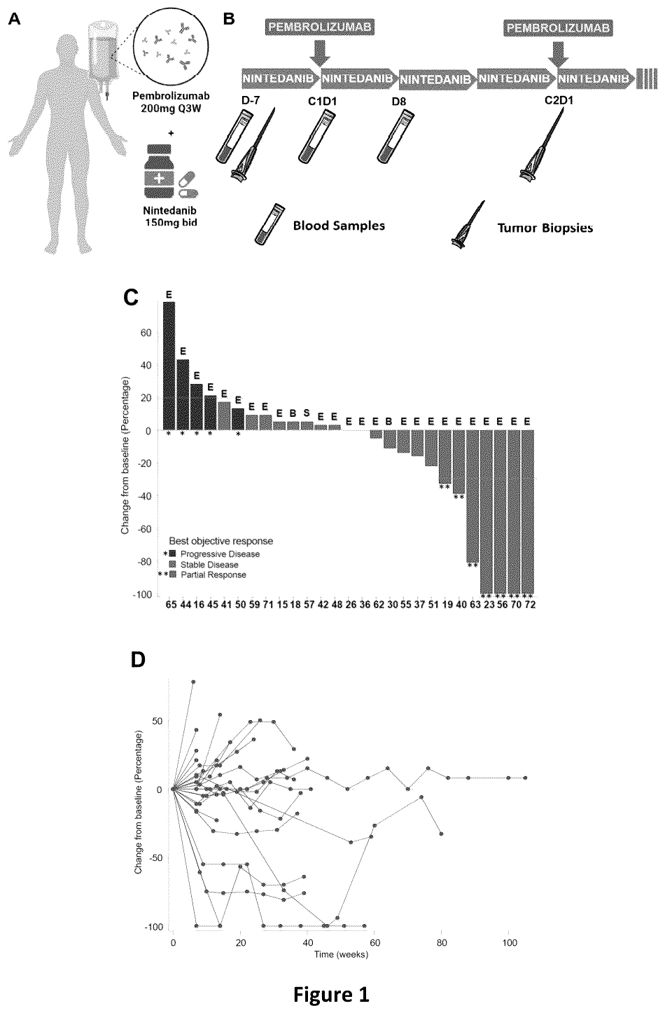

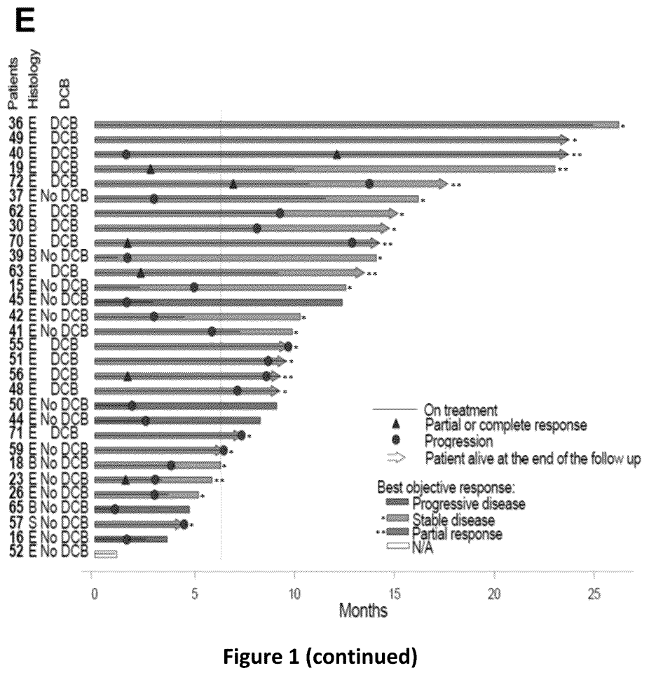

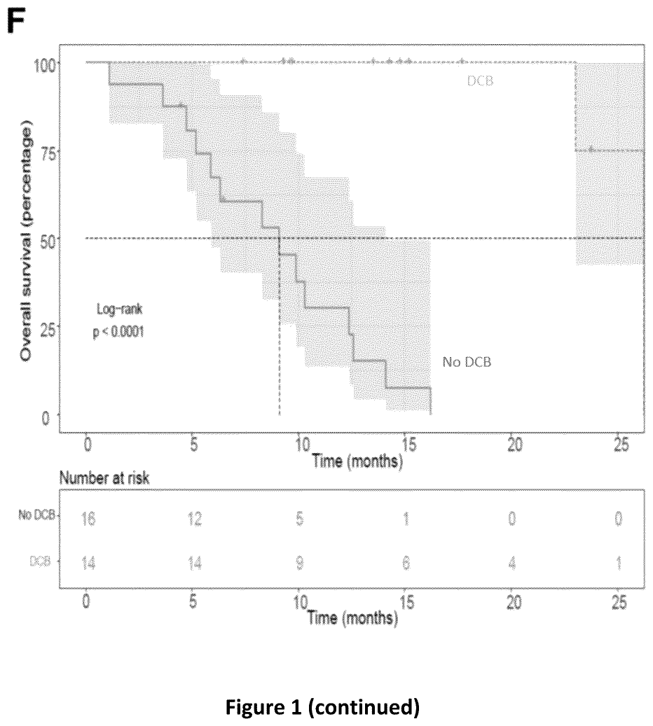

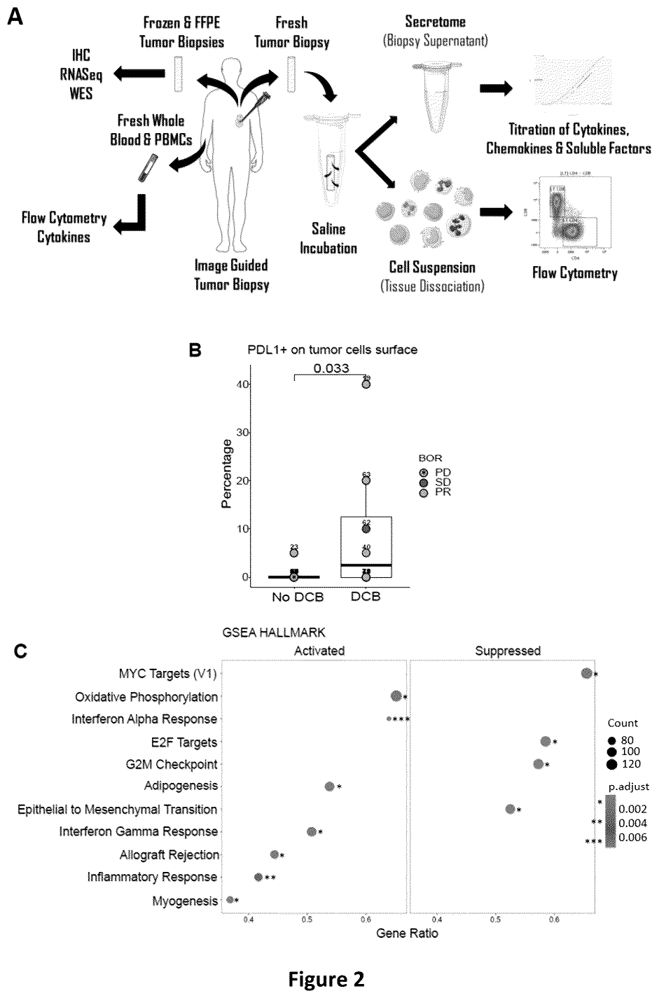

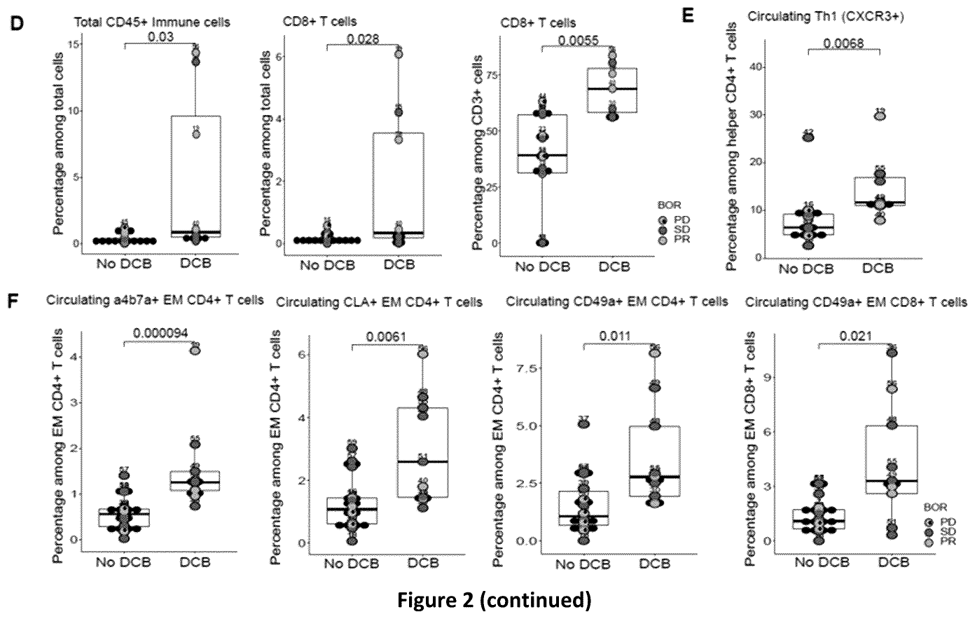

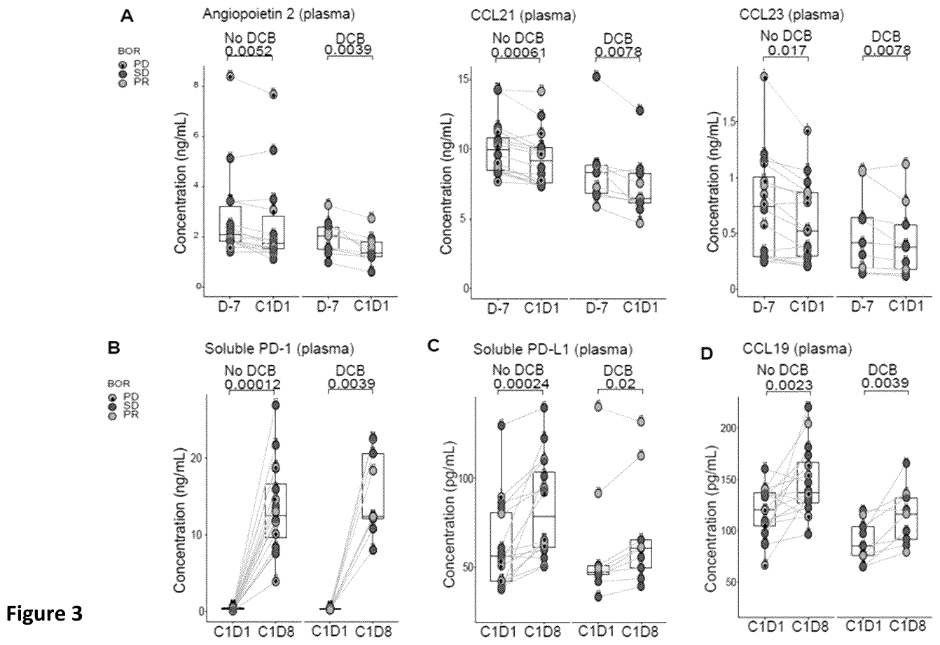

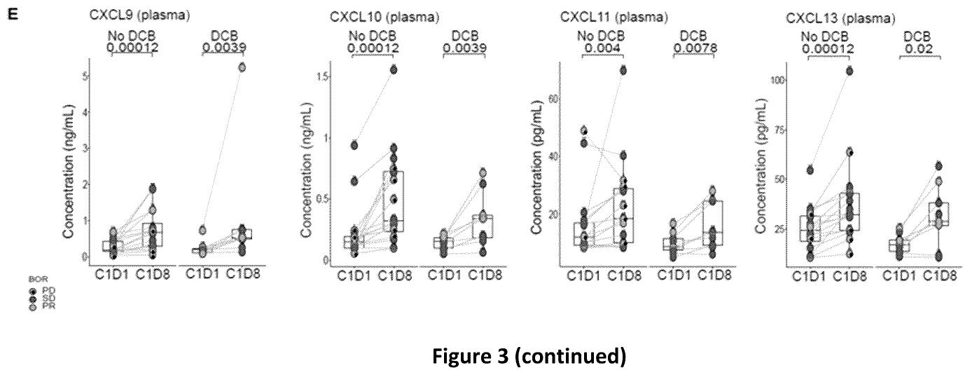

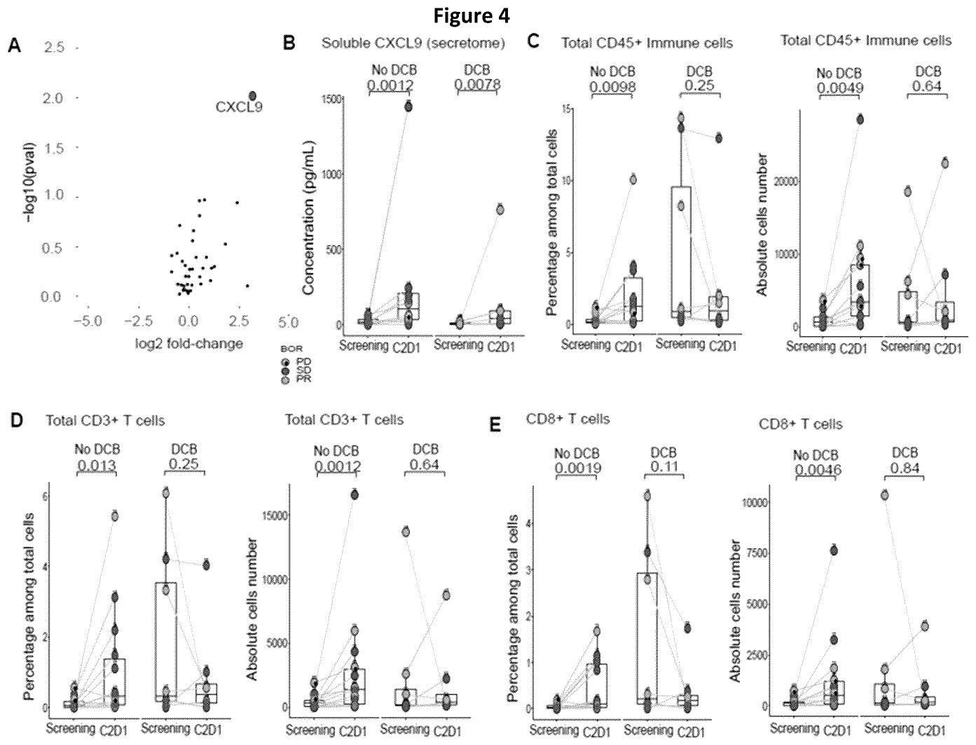

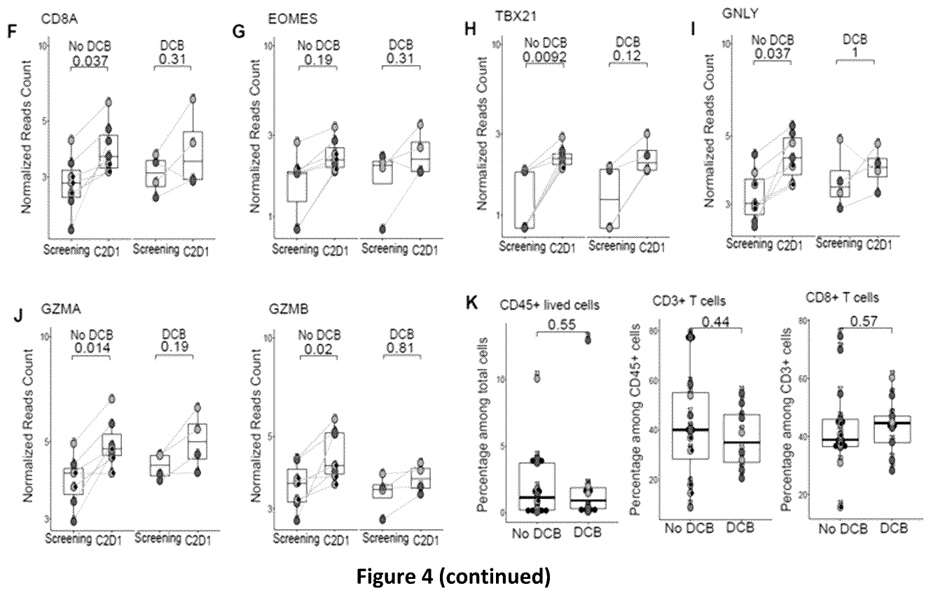

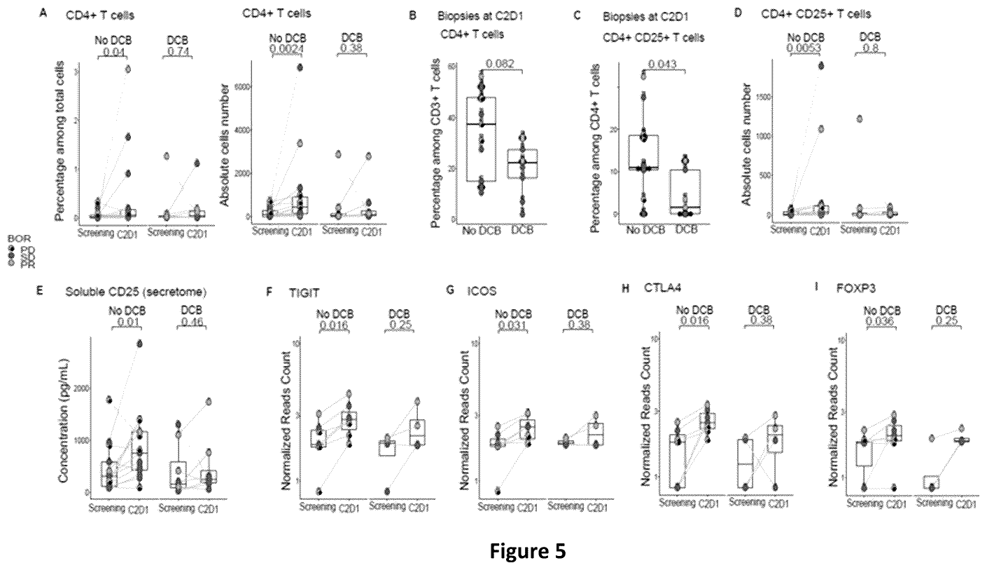

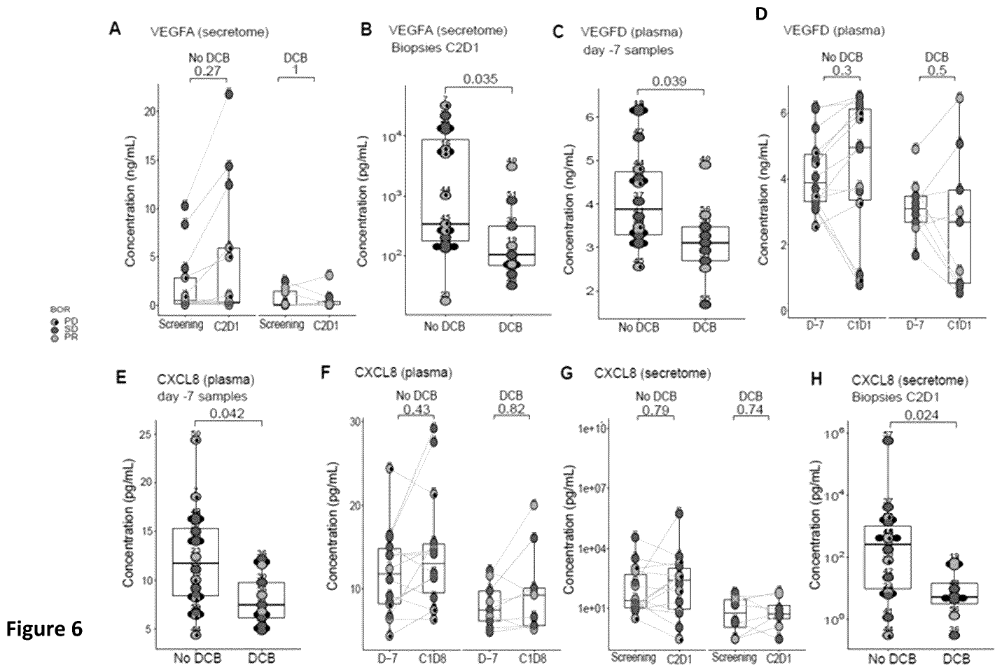

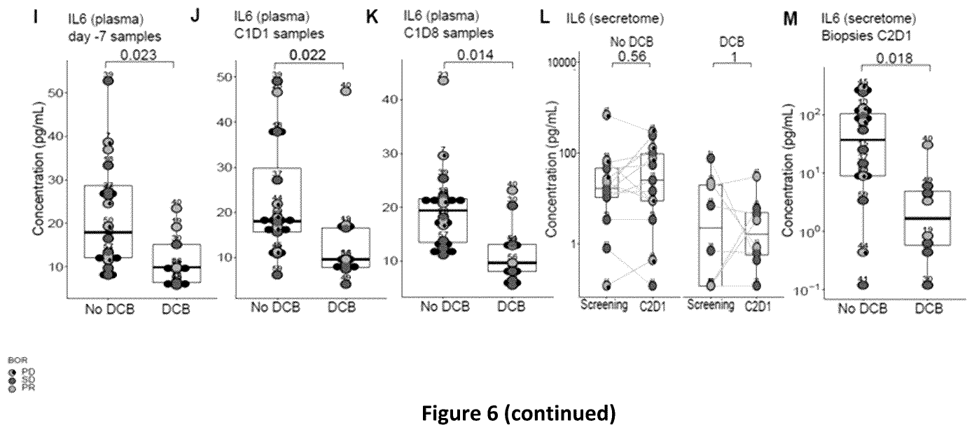

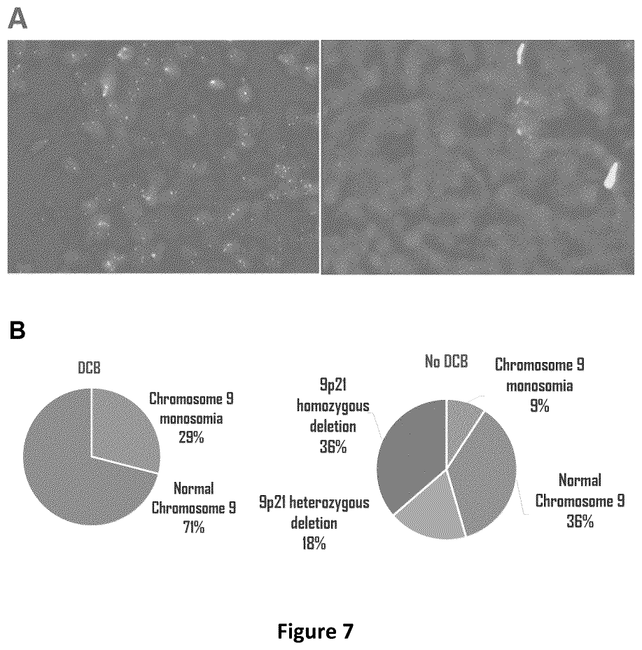

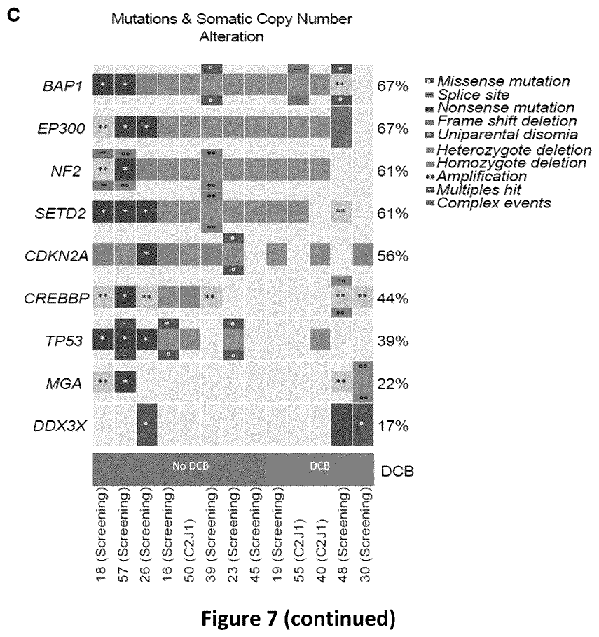

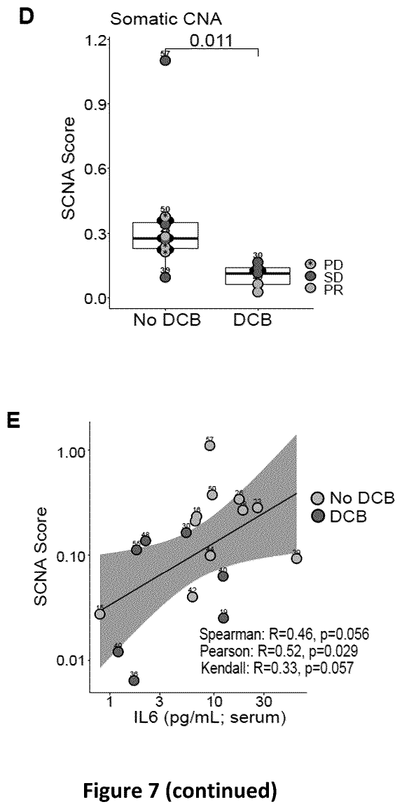

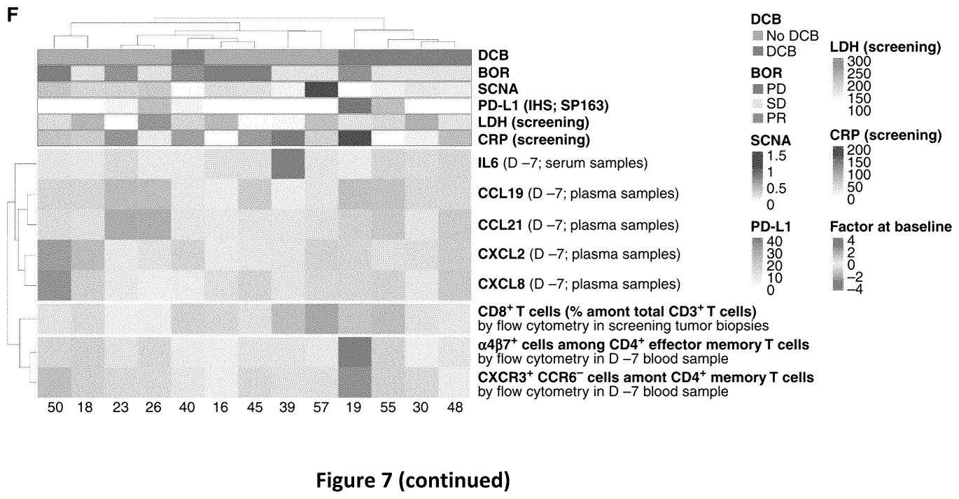

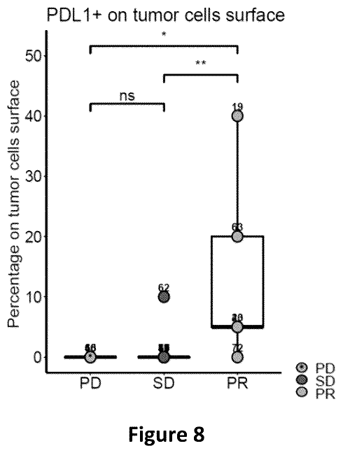

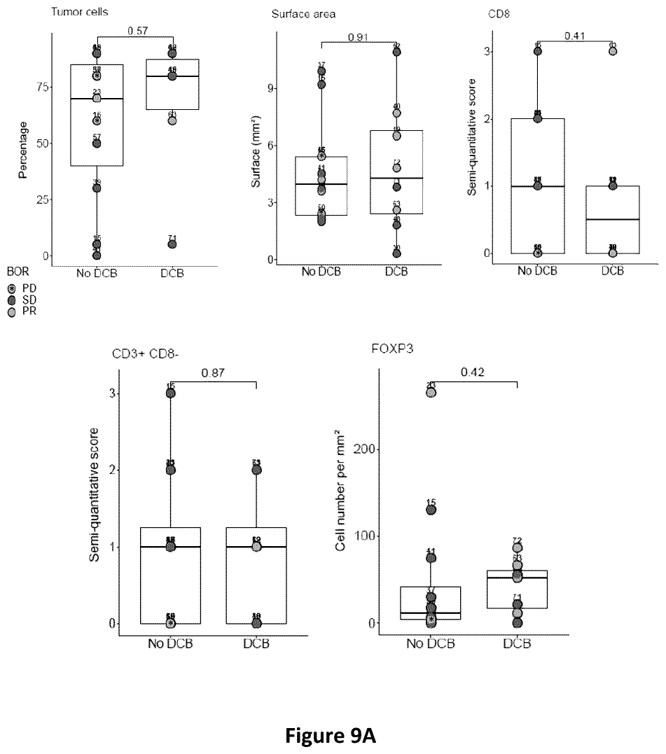

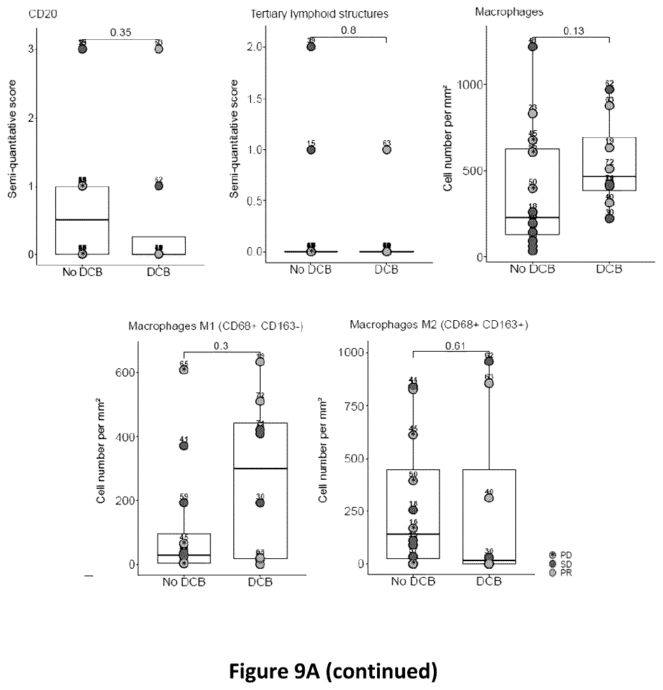

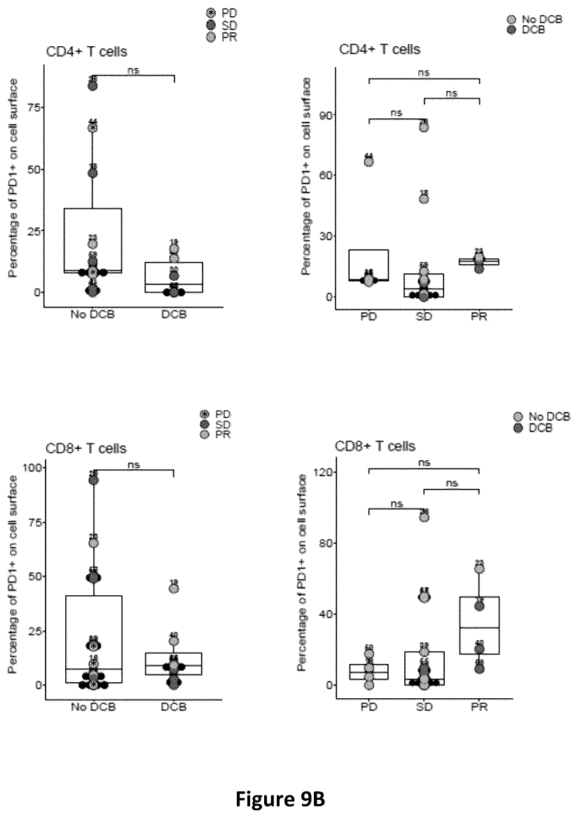

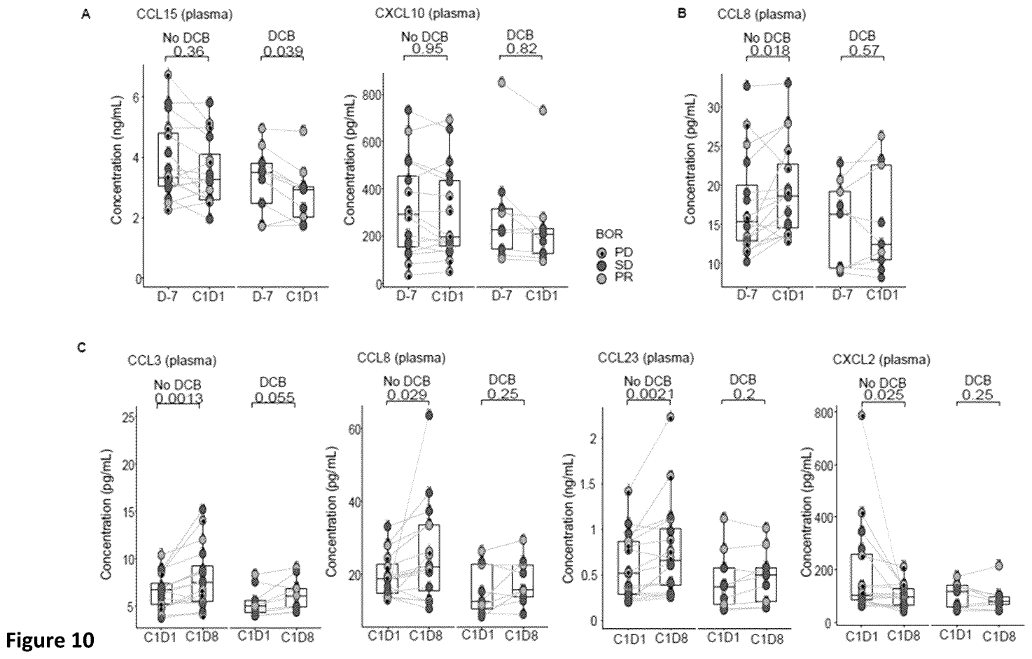

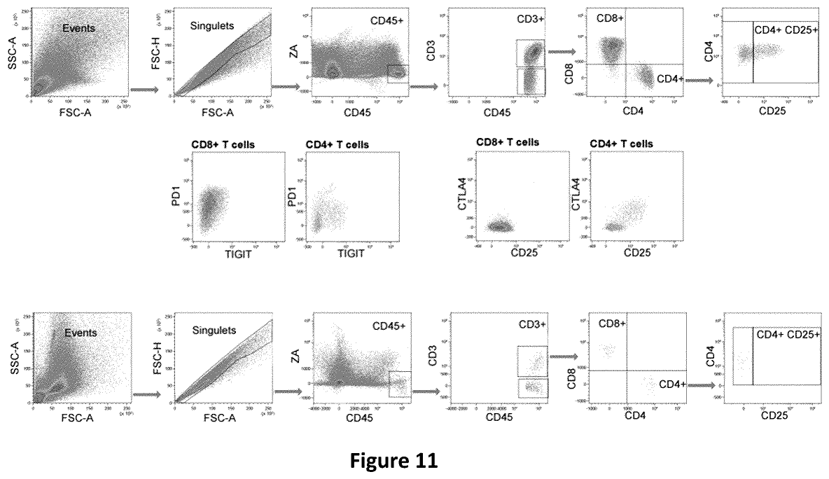

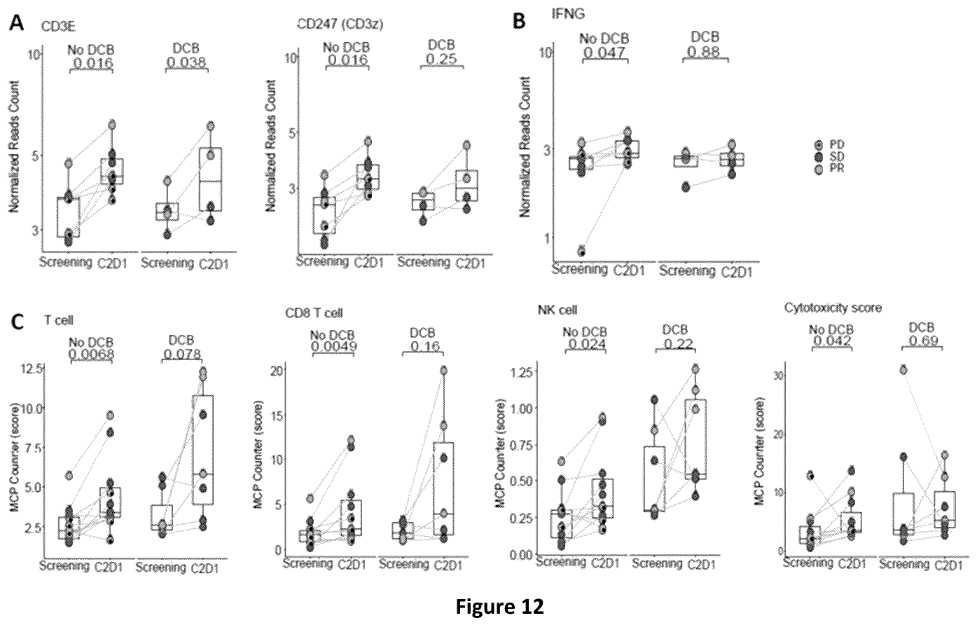

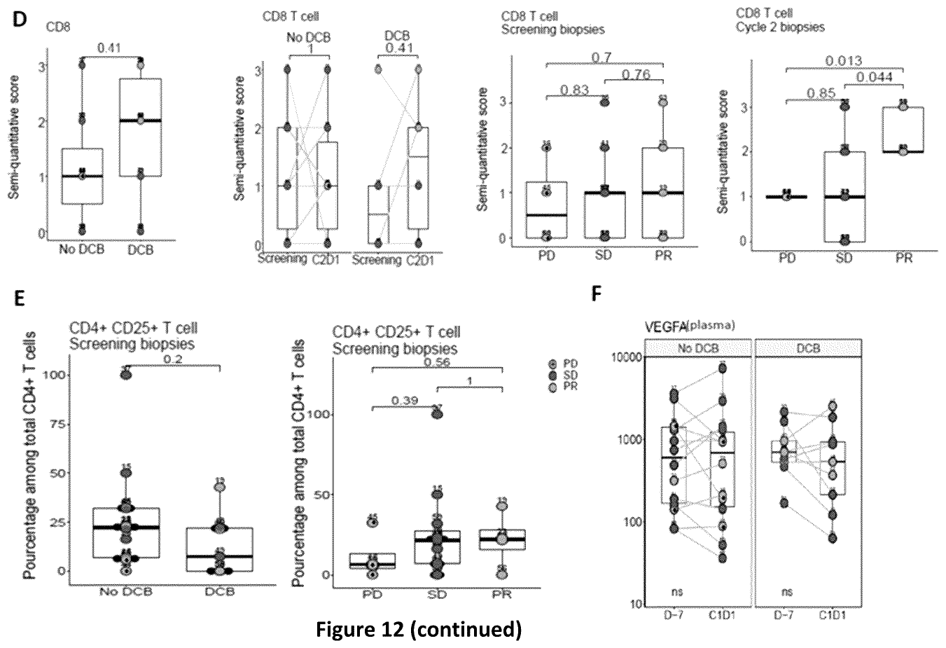

Other characteristics and advantages of the invention are given in the following experimental section (with reference to FIGS. 1 to 12), which should be regarded as illustrative and not limiting the scope of the present application.

### EXPERIMENTAL PART

### Example 1—Genomic Instability and Pro-Tumoral Inflammation are Associated with Primary Resistance to Anti-PD1+Anti-Angiogenesis in Malignant (or Advanced) (Pleural) Mesothelioma (MM)

Cancer immunotherapy combinations have been recently shown to improve the overall survival of advanced mesotheliomas especially for patients responding to those treatments. Inventors characterized the biological correlates of malignant pleural mesotheliomas primary resistance to immunotherapy and anti-angiogenics by testing the combination of pembrolizumab, an anti-PD-1 antibody, and nintedanib, a pan anti-angiogenic tyrosine kinase inhibitor (TKI), in the multi-center PEMBIB trial (NCT02856425). As described below, thirty patients with advanced malignant pleural mesothelioma were treated and explored. Unexpectedly, they found that refractory patients were actively recruiting CD45+CD3+CD8+ cytotoxic T-cells in their tumors through CXCL9 tumor release upon treatment. However, these patients displayed high levels of somatic copy number alterations in their tumors that correlated in particular with high blood and tumor levels of IL-6 and CXCL8. Those pro-inflammatory cytokines resulted in higher tumor secretion of VEGF and tumor enrichment in regulatory T-cells.

### Materials and Methods

### Study Design

In this expansion cohort of the phase 1b clinical trial PEMBIB (NCT02856425), inventors evaluated the association of nintedanib (150 ng twice a day) in combination with intravenous (IV) flat dose of pembrolizumab 200 mg over 30 minutes every 3 weeks. The posology of nintedanib was first determined in a dose escalation cohort, which showed that nintedanib at 150 mg BID was better tolerated than 200 mg BID, and subsequently selected for the expansion cohorts (Baldini C. et al.). Of note, patients received a one-week lead-in course of nintedanib monotherapy prior to starting pembrolizumab. The protocol was first approved by the Agence Nationale de Sécurité du Médicament (ANSM) on Jun. 24, 2016 (Ref #160371A-12). The protocol was also approved by the Ethical Committee (Comité de Protection des Personnes lie de France 1) on Jul. 12, 2016 (Ref #2016-mai-14236ND). The trial was first posted on clinicaltrials.gov on Aug. 4, 2016 (NCT02856425).

### Patients

Eligible patients had advanced pleural mesothelioma (“advanced MM”) who progressed after at least one line of standard therapy, naïve to immune checkpoint blockade and nintedanib. Additional inclusion criteria included age ≥18 years, Eastern Cooperative Oncology Group (ECOG) performance status of 0-1, adequate organ function, measurable disease according to RECIST v1.1 criteria, and written informed consent. Key exclusion criteria were radiographic evidence of cavitary tumors, local invasion of major blood vessels and/or at risk for perforation, history of clinically significant hemoptysis within the past 3 months, history of clinically significant hemorrhagic or thromboembolic event in the past 6 months, history of significant cardiovascular diseases, prior treatment with nintedanib and anti PD-(L)1 agents, concurrent steroid medication, history of autoimmune and inflammatory disease. This study was conducted in compliance with the Declaration of Helsinki and the International Ethical Guidelines for Biomedical Research Involving Human Subjects.

### Procedures

Screening procedures were performed up to 21 days (D-28) before Day −7 (start of nintedanib). Patients continued treatment until disease progression, undue toxicity, withdrawal of consent or for a maximum duration of 24 months. Adverse events were graded using National Cancer Institute (NCI) CTCAE Version 4.03. Tumor responses were evaluated every 6 weeks based on Response Evaluation Criteria in Solid Tumors (RECIST) 1.1 (41).

### Outcomes

The objectives of the trial were to determine the tolerability and safety of oral nintedanib 150 mg BID combined with IV pembrolizumab 200 mg Q3W and to evaluate the first efficacy signals with RECIST 1.1 best objective response rate (BOR), progression free survival (PFS), overall survival (OS) of this combination in a dedicated cohort of advanced MM. The aim of the ancillary studies was to identify predictive biomarkers of efficacy or resistance to this combination therapy.

Analyses of Tumor Infiltrating Immune Cells from Biopsies by Flow Cytometry

Fine-needle biopsy samples from tumoral lesions were immediately placed into 1 ml of NaCl 0.9%. After a minimum of 30 minutes of incubation, the supernatant of fresh tumor biopsies in 0.9% NaCl was collected and frozen at −80° C. and biopsies were mechanically dissociated with the top of a 2 ml syringe plunger in a wet 70 μm filter placed at the top of a 50 ml centrifuge tube. Isolated cells were washed by centrifugation and re-suspended in NaCl 0.9% for cell surface staining protocol. Cells were stained with anti-CD3/BUV395 (clone UCHT1; ref. 563546, BD Biosciences), anti-CD4/BUV496 (clone SK3, ref. 564651, BD Biosciences), anti-CD45/BUV805 (clone H130, ref. 612891, BD Biosciences), anti-PD1/BV421 (clone MIH4, ref. 564323, BD Biosciences), anti-OX40/BV650 (clone ACT35, ref. 563658, BD Biosciences), anti-CD39/FITC (clone TU66, ref. 561444, BD Biosciences), anti-HLA-DR/PerCP5.5 (clone G46-6, ref. 551764, BD Biosciences), anti-CTLA4/PF (clone BNI3, ref. 555853, BD Biosciences), anti-41BB/PECF594 (clone 4B4-1, ref. 309826, BD Biosciences), anti-CD25/PECy7 (clone B1.49.9, ref A52882, Beckmann Coulter), anti-TIGIT/APC (clone MBSA43, ref. 17-9500-41, Biolegend), anti-HLA-ABC/AF700 (clone W6/32, ref. 311438, Biolegend), anti-CD8/APC-H7 (clone SK1, ref. 560179, BD Biosciences) and Zombie Aqua™ Fixable Viability (ref. 423101, Biolegend). CTLA-4 was first stained at 37° C. for 20 min before other surface antibodies were added and incubated at 4° C. for 15 min. Then, cells were washed two times and acquired on a 18-colors flow cytometer BD Fortessa X20 (BD Biosciences). Data were acquired in FCS 3.0 format and analyzed with KALUZA software version 2.1.

### Immune Monitoring—Fresh Blood Immune Phenotype

Heparinized blood samples (30-40 mL) at day −7 (baseline), C1D1 and C5D1 were collected whenever possible for monitoring circulating immune populations by flow cytometry. Fresh whole blood phenotyping of T-cell migration, T-cell polarization, T-cell activation. Treg funetion and nyeloid cells was performed using 5 specific panels, as previously described (Pitoiset F. et al.). Stained cells were acquired using a Gallios Cytometer (Beckman Coulter) and analyzed using Kaluza software (Beckman Coulter).

### Cytokine, Chemokine and Soluble Angiogenic Factors Measurements

Frozen plasma and frozen tumor biopsy supernatants were subsequently thawed, centrifuged for 15 min at 1,000 g, then titrated using Bio-Plex Pro™ Human Chemokine Panel (40-Plex, ref. 171AK99MR2, Bio-rad), Angiogenesis Panel 1 (human) (ref. K151P3S-1, Meso Scale Discovery), Human PD-1 and PD-L1 antibody sets (ref. F214A-3 & F214C-3, Meso Scale Discovery) following manufacturer's instructions. Each sample was run twice with the average value of the doublet taken as the result. Acquisitions were done on Bio-Plex 200™ System and MESO™ QuickPlex SQ120 readers. Raw data from Meso Scale Discovery's kit were analyzed with MSD's Discovery Workbench 4.0.

Serum IL6 quantifications were subsequently confirmed with Siemens Atellica IM1600 and Atellica IM Interleukin-6 kit (Siemens Healthineers, Saint-Denis, France) and validated by the Gustave Roussy accredited biochemistry diagnostic laboratory. Quantification method validation was performed according to ISO15189 recommendation. The quantification range covers from 2.7 (limit of quantification) to 5500 pg/ml. Any quantification batches included 3 levels of internal quality control analysis. IL6 titrations obtained by Siemens were well correlated to the ones obtained on Bio-Rad.

### Immunohistochemistry

Immunohistochemical (IHC) chromogenic staining were performed on formalin fixed paraffin embedded (FFPE) tumor biopsies. PD-L1 (staining was performed using the SP263 PD-L1 assay) single chromogenic staining were performed using the Ventana Benchmark Ultra platform. CD20 staining and CD3 (Purple)/CD8 (DAB) chromogenic dual staining were performed using the Ventana Discovery Ultra platform. Tumor areas were selected by a senior pathologist blinded from the outcome of the patients.

### Fluorescent In Situ Hybridization

Fluorescent In Situ Hybridization (FISH) tests were performed on formalin-fixed paraffin-embedded (FFPE) tumor tissues using ZytoLight SPEC CDKN2A/CEN 9 Dual Color Probe according to the manufacturer's instructions (ZytoVision, Bremerhaven, Germany). The SPEC CDKN2A/CEN 9 Dual Color Probe is a mixture of a red fluorochrome directly labeled CEN 9 probe specific for the classical satellite III region of chromosome 9 (D9Z3) at 9q 12 and a green fluorochrome directly labeled SPEC CDKN2A probe specific for the CDKN2A gene at 9p21.3. Slides were deparaffinized and then preincubated with the pretreatment buffer (citric solution) at 98° C. during 15 min followed by protease treatment 15 min at 37° C. After washes and dehydration with ethanol, slides were denatured at 75° C. during 10 min then hybridized with the probe overnight at 37° C. After several washes the following day, counterstain was added onto the slides (4′6-diamidino-2-phenylindole (DAPI)). 50 cells were counted on each tumor sample categorizing signals as follows, counting the number of green signal and red signals in each cell: loss of one green signal and normal red signals (loss of one copy of CDKN2A); loss of two green signals and normal red signals (loss of two copies of CDKN2A); loss of one green signal and one red signal (loss of a whole chromosome 9 or short arm); normal red and green signals (normal cell).

### Whole-Transcriptome RNA-Seq

Integrity (RNA Integrity Score≥7.0) of RNA extracted from frozen tumoral biopsies was checked on the Agilent 2100 Bioanalyzer (Agilent) and quantity was determined using NanoDrop (Thermofisher Scientific). SureSelect Automated Strand Specific RNA Library Preparation Kit was used according to the manufacturer's instructions with the Bravo Platform. Briefly, 50 to 200 ng of total RNA sample was used for poly-A mRNA selection using oligo (dT) beads and subjected to thermal mRNA fragmentation. The fragmented mRNA samples were used for cDNA synthesis and were further converted into double stranded DNA using the reagents supplied in the kit, and the resulting dsDNA was used for library preparation. The final libraries were bar-coded, purified, pooled together in equal concentrations, and processed for paired-end 2×100 sequencing on Novaseq-6000 sequencer (Illumina) at Gustave Roussy.

### Bulk Tumor RNA-Seq Analyses

The QC and analysis pipeline was based on Love et al., powered by SnakeMake (Love M I et al., 2018; Koster J et al.). Quality controls were performed on raw FastQ files with FastQC v0.11.9. Reads trimming for low 3′ terminal base quality and removing of adapter sequences was performed using fastp v0.20.1. Sample contamination was assessed with FastqScreen v0.14.0. Quality reports were gathered with MultiQC v1.9 (Ewels P. et al.). Abundance estimation was performed with Salmon v1.4.0, using 100 bootstraps and the GenCode v34 annotations, corresponding to the GRCh38 genome build (Patro R. et al.). Aggregation was performed with the tximport package, and differential gene analysis was performed with DESeq2, with a formula design taking care of the sample effect when sample pairs (Screening/C2J1) were taken into consideration (Love M I. et al., 2014). Deconvolution of immune cell fractions from bulk RNA sequencing data was done with immunedeconv package (Sturm G. et al.). Gene set enrichment and overall representation analyses (GSEA/ORA) were performed with the clusterProfiler package v4.0.2, against the MsigDb collections, the Disease Ontology, and the KEGG, CellMarker and MeSH databases (Yu G. et al.). For quality control, the variance stabilizing transformation (vst) normalization from DESeq2 was applied on raw counts. Of note, RNA-seq data from baseline biopsies without tumor cells were not integrated in the analyses. Also, the data from patient #23 baseline pleural biopsy, who had a peritoneal progression before 6 months, was considered in the durable clinical benefit (“DCB”) group because of a primary and persistent complete response of pleural lesions.

### Somatic and Germline Whole Exome Sequencing (WES)

50 to 200 ng of genomic DNA was extracted from frozen tumor biopsies with the Covaris E220 system (LGC Genomics/Kbioscience). DNA fragments were end-repaired, extended with an ‘A’ base on the 3′ end, ligated with paired-end adaptors with the Bravo Platform (Agilent) and amplified (ten cycles). Exome-containing adaptor-ligated libraries were hybridized for 40 h with biotinylated oligo RNA baits using SureSelect Clinical Research 2 (Agilent) and enriched with streptavidin-conjugated magnetic beads. The final libraries were indexed, pooled, and sequenced using the onboard cluster method, as paired-end sequencing (2×100 bp reads) on Illumina NovaSeq-6000 sequencer at Gustave Roussy.

### Whole Exome Sequencing Analyses

Identification of mutations and individual somatic copy number alteration were done with the following methods. Reads were mapped using the BW A-MEM (v0.7.12) software (Li H. et al.) to the GRCh37 hunman reference genome and then inventors used the standard GATK best practice pipeline (Van der Auwera G A, et al.) to process the samples and call somatic genetic variants. PCR duplicates were removed, and base quality score recalibrated using MarkDuplicates and BaseRecalibrator tools which a part of the GATK package (DePristo M A et al.). Somatic SNVs and INDELs were called and filtered using GATK tools Mutect2, FilterMutectCalls and FilterByOrientationBias and annotated with oncotator (Ramos A H et al.). Quality controls of FASTQ and mapping were done with FASTQC, samtools (v1.9), GATK HSmetrics and multiqc (Ewels P. et al.; Li H. et al.). The processing steps were combined in a pipeline built with snakemake (Köster J. et al.). Somatic mutations with PASS flag from GATK Mutect2 were additionally filtered to have at least 1 supporting reads from each strand and 3 reads in total. Inventors used then MAF annotator to find oncogenic mutations from OncoKB database and visualized them as an oncoplot with the maftools R package (Mayakonda A. et al.). Tumor mutational burden of the samples was calculated in accordance with the guidelines proposed by the “Friends of Cancer Research TMB Harmonization Project” (Merino D M et al.). Identification of copy-number alterations were performed using EaCoN v0.3.6 (https://github.com/gustaveroussy/EaCoN) on R v3.6.2. Per-patient paired samples were analyzed using tumoral samples as test, and genomic DNA from PBMCs used as reference, for each pair. GATK-recalibrated BAM files were transformed to the mpileup format using Rsamtools v2.8.0, ignoring replicates and secondary alignments (Morgan M. et al.). Depth of each nt for test and reference was computed using mpileup, then binned to windows of 50 nt in median (depending on the capture BED information). Bins with a total depth <20 were discarded. Using a pre-generated track of GC % content in bins, those with a value <20% or >80 were identified as outliers. Still for each bon, median depths were converted to log 2(Test/Ref) (L2R), and L2R for GC % outliers was imputed using kNN. L2R was then normalized for (GC % using a lowess regression. To generate the BAF data, any non-reference sequences in the mpileups were identified and their depth quantified (Van Loo P. et al.). SNP variants supported by less than 3 reads and/or for which the total depth was below 20 were discarded. All SNP variants in the test sample with a reference frequency below 33% were discarded. The bivariate (L2R and BAF) data were then segmented, evaluated for their allele-specific absolute copy-number, as well as ploidy and tumor cellularity, using ASCAT v2.5.2 (https://github.com/VanLoo-lab/ascat/releases).

The somatic copy number alteration score (“SCNA score” or “SCNA genomic instability score”) was established as follow: for each sample, the absolute copy number (ACN) profile generated by ASCAT through EaCoN was used. For each chromosome taken independently, the basal ACN level was identified as the one with the longest total width. Then, for each chromosome, ACN levels were converted into the absolute difference in copy number (aDCN) to this basal level (i.e., if basis was 3 copies, a 1 copy segment value will be 2; 2 copies==>; 3 copies 0; 4 copies=>1; etc.). The final SCNA LOCAL score was computed as the width-ponderated sum of each of these converted CN-to-basis values, divided by the total covered genome length.

### Statistical Analysis and Illustrations

Clinical statistical analysis has been done using the SAS® statistical software version 9.4 (Cary, North Carolina, USA). Calculations and statistical tests for ancillary analyses were performed using R v3.4. Wilcoxon-Mann-Whitney test was used to assess differences between two patient's groups. Data representation and analyses were performed with software R v3.3.3 using tidyverse, dplyr, ggplot2, ggpubr, complexheatmap, survival and atable packages. Figure's aesthetics were worked with Affinity Designer® (v1.9.2.1035). Statistical tests applied to ancillary analysis were exploratory. P-values were adjusted for multiple comparisons only for the differential gene expression analysis in transcriptomic data from screening tumor biopsies between patients with or without durable clinical benefit (DCB). All others p-values in the manuscript were not adjusted for multiple comparisons

### Results

### Trial Design & Patients Characteristics

The PEMBIB trial is a multicenter Phase 1b trial which consisted in a dose-escalation part to determine the recommended dose of nintedanib to be used with pembrolizumab, followed by expansion cohorts in disease specific indications. Patients treated in the expansion cohorts received Nintedanib at 150 mg orally bid in combination with Pembrolizumab at 200 mg IV Q3W (FIG. 1A). A Nintedanib monotherapy lead-in was initiated seven days (D-7) before initiating Pembrolizumab at C1D1 (FIG. 1B). Here inventor report the results of the first expansion cohort completed with patients having advanced (pleural) MM. Thirty-two patients were enrolled between Oct. 10, 2017, and Apr. 11, 2019, in the mesothelioma expansion cohort of the trial. Two patients were screen failed because of exclusion criteria and n=30 patients were finally treated in the trial. Baseline characteristics of the patients are described in below Table 1.

TABLE 1
Baseline characteristics of the patients treated

| | Total (n = 30) | |
|---|---|---|
| Sex | | |
| Female | 10 | (33%) |
| Male | 20 | (67%) |
| Mean age, years [SD] | 69 | [11] |
| Body mass index, kg/m², mean [SD] | 25 | [4.9] |
| ECOG performans status | | |
| 0 | 9 | (30%) |
| 1 | 20 | (67%) |
| Missing | 1 | (3.3%) |
| Localization | | |
| Pleural mesothelioma | 30 | (100%) |
| Presence of Peritoneal carcinomatosis | 6 | (20%) |
| Histology subtypes | | |
| Epithelioid | 25 | (83%) |
| Biphasic | 4 | (13%) |
| Sarcomatoid | 1 | (3.3%) |
| TNM UICC (v.8) | | |
| III | 20 | (67%) |
| IV | 10 | (33%) |
| Previous systemic anticancer treatment | | |
| 1 | 23 | (77%) |
| 2 | 5 | (17%) |
| ≥3 | 2 | (6.7%) |
| Previous treatment with Bevacizumab | | |
| No | 18 | (60%) |
| Yes | 12 | (40%) |
| BAP1 expression status (IHC) | | |
| Loss | 9 | (30%) |
| Normal | 3 | (10%) |
| Not done | 12 | (40%) |
| Missing | 6 | (20%) |
| PDL1 expression on tumor cells | 4.1 | [9.5] |
| (IHC), mean [SD] | | |
| Median leucocytes (.10³/mm³) (range) | 8.55 | (3.6-15) |
| Neutrophils to lymphocytes ratio, | 4.5 | [1.9] |
| mean [SD] | | |
| Median ASAT (UI/L) (range) | 25 | (14-48) |
| Median LDH (UI/L) (range) | 187.5 | (130-622) |
| Median Albumin (g/L) (range) | 39 | (29-46) |
| Median CRP (mg/L) (range) | 37.8 | (2.4-171.5) |
| Median IL6 (pg/mL) (range) | 6.7 | (0.8-62) |

All patients enrolled were initially treated for primary pleural mesothelioma. However, 6 of them (Patients #19, 23, 45, 50, 59 and 71) had also a peritoneal involvement of mesothelioma at the time of enrollment. Among them, two patients had a partial response. But one patient only responded at his pleural site, and progressed at his peritoneal site after 4 months of treatment; he has been considered as “No DCB” patient per definition. Two had a stable disease as best objective response (one “DCB” and the other “No DCB” ) and the two last patients had progressive diseases and “No DCB”. Membrane PD-L1 expression by cancer cells was assessed by immuno-histo-chemistry using the anti-PDL1 staining clone SP263 and following the TPS scoring methodology on baseline tumor biopsies. Tumors were considered positive for PD-L1 if they had more than a TPS of 1%. Abbreviations: SD = standard deviation; ECOG = Eastern Cooperative Oncology Group; UICC = International Union against Cancer; BAP1 = BRCA1 Associated Protein; IHC = Immuno-Histo-chemistry; PDL1 = Programmed Death Ligand 1.

The patients enrolled presented advanced tumors involving the pleura, with metastatic peritoneal carcinomatosis in 6/30 cases (20%) and were all refractory to or relapsing after first line platinum-based doublet chemotherapy. The number of previous lines of treatment were 1, 2 and ≥3 for 23/30 (77%), 5/30 (17%) and 2/30 (6.7%) of the patients, respectively. Previous treatment with bevacizumab in combination with chemotherapy was reported for 12/30 (40%). Dose and/or scheduling modifications occurred in 12/29 (41%), and 4/29 (14%) of the patients because of adverse events associated with nintedanib and pembrolizumab, respectively. Treatment and dose modifications have been summarized in below Table 2.

TABLE 2
Treatment and dose modifications in
patients according to their DCB status.

| | DCB | No DCB | |
|---|---|---|---|
| | (n = 14) | (n = 16) | p |
| Treatment modification | 8 (57%) | 10 (62%) | 1 |
| Drug modification | | | |
| pembrolizumab & nintedanib | 3 (21%) | 3 (19%) | 0.81 |
| suspension | | | |
| nintedanib suspension | 5 (36%) | 6 (38%) | |
| pembrolizumab suspension | 0 | 1 (6.2%) | |
| Reason of suspension | | | |
| Adverse events | 8 (100%) | 6 (60%) | 0.53 |
| Disease progression | 0 | 1 (10%) | |
| Infection disease | 0 | 1 (10%) | |
| Patient decision | 0 | 1 (10%) | |
| Patient misunderstanding | 0 | 1 (10%) | |
| Rechallenge | 8 (100%) | 6 (60%) | 0.12 |
| Treatment adaptation | | | |
| nintedanib dose reduction | 8 (100%) | 5 (50%) | 0.23 |
| nintedanib continuation | 0 | 1 (10%) | |
| End of nintedanib | 0 | 2 (20%) | |
| End of treatments | 0 | 2 (20%) | |

Treatment emergent adverse events related to the study drugs per clinical investigator assessment are reported in below Table 3.

TABLE 3
Adverse events emergent during treatment
and related to experimental drugs by the
investigators according to CTCAE Version 4.03.

| | Grade 1-2 | | Grade 3 | Grade 4 | Grade 5 |
|---|---|---|---|---|---|
| Alanine | 3 | (10%) | 0 | 0 | 0 |
| aminotransferase | | | | | |
| increased | | | | | |
| Anemia | 4 | (13.3%) | 0 | 0 | 0 |
| Arthralgia | 6 | (20%) | 0 | 0 | 0 |
| Aspartate | 3 | (10%) | 0 | 0 | 0 |
| aminotransferase | | | | | |
| increased | | | | | |
| Central nervous system | 5 | (16.7%) | 0 | 0 | 0 |
| disorder* | | | | | |
| Colitis | | 0 | 1 (3.3%) | 0 | 0 |
| Cough | 7 | (23.3%) | 0 | 0 | 0 |
| CPK increased | 2 | (6.7%) | 0 | 1 (3.3%) | 0 |
| Creatinin increased | 1 | (3.3%) | 0 | 0 | 0 |
| Decreased appetite | 6 | (20%) | 1 (3.3%) | 0 | 0 |
| Diarrhea | 18 | (60%) | 1 (3.3%) | 0 | 0 |
| Dyspnea | 11 | (36.7%) | 2 (6.7%) | 0 | 0 |
| Dysphagia & dyspepsia | 3 | (10%) | 0 | 0 | 0 |
| Fatigue | 14 | (46.7%) | 2 (6.7%) | 0 | 0 |
| Fever | 6 | (20%) | 0 | 0 | 0 |
| GGT increased | 1 | (3.3%) | 1 (3.3%) | 0 | 0 |
| Hypomagnesemia | 5 | (16.7%) | 0 | 0 | 0 |
| Hypothyroidism | 4 | (13.3%) | 0 | 0 | 0 |
| Lipase increased | 1 | (3.3%) | 2 (6.7%) | 1 (3.3%) | 0 |
| Mucositis | 1 | (3.3%) | 0 | 0 | 0 |
| Myocarditis & cardiac | 1 | (3.3%) | 1 (3.3%) | 0 | 1 (3.3%) |
| disorder | | | | | |
| Nausea | 7 | (23.3%) | 1 (3.3%) | 0 | 0 |
| Peripheral nervous | 2 | (6.7%) | 0 | 0 | 0 |
| system disorder** | | | | | |
| Pneumonitis | 3 | (10%) | 0 | 0 | 0 |
| Skin disorder (including | 6 | (20%) | 2 (6.7%) | 0 | 0 |
| rash & pruritis) | | | | | |
| Vomiting | 10 | (30%) | 0 | 0 | 0 |
| Weight loss | 3 | (10%) | 0 | 0 | 0 |

The most frequent adverse events (AE) (grades 1-3) related to the combination therapy were diarrhea, fatigue, nausea, and liver enzymes elevation. Twelve (40%) and 3/30 (10%) patients developed grade 3/4/5 treatment- and immune-related adverse events respectively (colitis with pneumonitis [n=1] and myocarditis [n=2], grade 3, 4 and 5, respectively). Two patients developed arterial thrombosis (acute coronaropathy [n=2] and mesenteric ischemia [n=1]). Patients died because of cancer progression (n=14/30, 46.7%), cardiopathy resulting to thrombosis and mesenteric ischemia which have been related to treatment (n=1/30, 3.3%) and COVID-19 (n-=/30, 3.3%).

### Efficacy of Pembrolizuinab+Nintedanib in Pleural Mesothelioma

The median follow-up of the cohort at database lock was 14.8 months (95% confidence interval [95% CI][9.70-8.2]). The median progression free survival (PFS) was 6.2 months (95% CI [3-8.7]). At database lock, the median Overall Survival (OS) was 14.1 months (95% CI [9.89-Not Reached]). The median OS for patients with No durable clinical benefit (DCB) was 9.1 month (95% CI [5.88-14.1]), and 26.3 months for patients with DCB (95% CI [23.03-NA]). One patient could not be evaluated for tumor response because of early death related to the above-mentioned grade 5 adverse event. Best Objective Responses (BOR) per RECIST1.1 criteria were Partial Response (PR, n=7/29; 24.1%). Stable Disease (SD, n=17/29; 58.6%) and Progressive Disease (PD, n=5/29; 17.2%) (FIG. 1C). Disease Control Rates (DCR) (defined as PR+SD) were 68.4% (95% IC [43.4:87.4]) and 46.6% at 3 and 6 months, respectively. At database lock, two patients (7%) ended treatment because they completed the 2-year treatment per protocol but 23/30 (79%) had to stop because of cancer progression. Some patients presented durable tumor responses or durable stable disease (FIG. 1D). Therefore, for ancillary analysis, inventors decided to classify patients as having Durable Clinical Benefit (i.e., RECIST1.1 PR or SD at 6 months post C1D1; further called “DCB patients”), or having No Clinical Benefit (i.e., RECIST1.1 PD before 6 months post C1D1; further called “No DCB patients”) (FIG. 1E). The baseline characteristics of the patients treated according to their DCB status is provided in below Table 4.

TABLE 4
Baseline characteristics of the patients
treated according to their DCB status.

| | DCB | | No DCB | | |
|---|---|---|---|---|---|
| | (n = 14) | | (n = 16) | | p |
| Sex | | | | | |
| Female | 4 | (29%) | 6 | (38%) | 0.9 |
| Male | 10 | (71%) | 10 | (62%) | |
| Mean age, years [SD] | 70 | [8.7 | 68 | [13 | 0.85 |
| Body mass index, | 25 | [5.4] | 25 | [4.6] | 0.74 |
| kg/m², mean [SD] | | | | | |
| ECOG performans status | | | | | |
| 0 | 10 | (71%) | 10 | (62%) | 0.61 |
| 1 | 4 | (29%) | 5 | (31%) | |
| Missing | | 0 | 1 | (6.2%) | |
| Localization | | | | | |
| Pleural mesothelioma | 14 | (100%) | 16 | (100%) | 0.78 |
| Presence of Peritoneal | 2 | (14%) | 4 | (25%) | |
| Mesothelioma | | | | | |
| Histology subtypes | | | | | |
| Epithelioid | 13 | (93%) | 12 | (75%) | 0.38 |
| Biphasic | 1 | (7.1%) | 3 | (19%) | |
| Sarcomatoid | | 0 | 1 | (6.2) | |
| TNM UICC (v.8) | | | | | |
| III | 9 | (64%) | 11 | (69%) | 1 |
| IV | 5 | (36%) | 5 | (21%) | |
| Previous systemic | | | | | |
| anticancer treatment | | | | | |
| 1 | 11 | (79%) | 12 | (75%) | 0.55 |
| 2 | 2 | (14%) | 3 | (19%) | |
| ≥3 | 1 | (7.1%) | 1 | (6.2%) | |
| Previous treatment | | | | | |
| with Bevacizumab | | | | | |
| No | 8 | (57%) | 10 | (62%) | 1 |
| Yes | 6 | (43%) | 6 | (38%) | |
| BAP1 expression | | | | | |
| status (IHC) | | | | | |
| Loss | 5 | (36%) | 4 | (25%) | 0.11 |
| Normal | 1 | (7.1%) | 2 | (12%) | |
| Not done | 3 | (21%) | 9 | (56%) | |
| Missing | 5 | (36%) | 1 | (6.25%) | |
| PD-L1 positivity on | 4/8 | [50%] | 1/12 | [12%] | 0.035 |
| tumor cells | | | | | |
| (IHC), # of patients [%] | | | | | |
| Median leucocytes | 8 | (3.6-12.2) | 10.1 | (4.5-15) | 0.58 |
| (.10³/mm³) (range) | | | | | |
| Neutrophils to | 4.8 | [2.1 | 4.3 | [1.8] | 0.88 |
| lymphocytes | | | | | |
| ratio, mean [SD] | | | | | |
| Median ASAT | 23 | (14-35) | 28 | (15-48) | 0.43 |
| (UI/L) (range) | | | | | |
| Median LDH | 172 | (130-266) | 197 | (132-622) | 0.33 |
| (UI/L) (range) | | | | | |
| Median Albumin | 39 | (29-45) | 38 | (35-46) | 0.99 |
| (g/L) (range) | | | | | |
| Median CRP | 10.7 | (2.4-171.5) | 53.4 | (4.6-142.5) | 0.23 |
| (mg/L) (range) | | | | | |
| Median IL6 | 5.3 | (1.2-12.3) | 9.2 | (0.8-62) | 0.047 |
| (pg/mL) (range) | | | | | |

The test used to compare nominal measurements was χ² test. Membrane PD-L1 expression by cancer cells was assessed by immuno-histo-chemistry using the anti-PDL1 staining clone SP263 and following the TPS scoring methodology on baseline tumor biopsies. Tumors were considered positive for PD-L1 if they had more than a TPS of 1%.

Inventors found that the 16 MM patients (53.3%) with No DCB per radiological assessment had a very bad outcome compared to the 14 MM patients (46.7%) with radiological DCB as illustrated by their drastic differences in overall survival (OS) upon Pembrolizumab+Nintedanib therapy (p<0.0001; FIG. 1F).

### Predictive Biomarkers of Favorable Outcome

All patients enrolled in the PEMBIB trial consented to undergo blood draws and tumor biopsies at baseline and on-treatment (FIG. 1B). Up to two cores of tumor biopsies were frozen and used for subsequent whole exome sequencing (WES) and bulk gene expression analysis (RNA seq). Up to two cores of tumor biopsies were formalin-fixed and paraffin embedded for subsequent immunohistochemistry (IHC) stainings. Up to two cores of fresh tumor biopsies were immediately put into physiological medium. Those biopsies were monitored for cytokine and soluble factor releases and then turned into cell suspension for flow cytometry analysis (FIG. 2A). Inventors prospectively analyzed the samples to characterize the biology of patients with No DCB and identified biomarkers associated with primary resistance to Pembrolizumab+Nintedanib.

First, inventors aimed to confirm the value of the two biomarkers that have been previously associated with efficacy of checkpoint blockade immunotherapies in mesothelioma. Looking at tumor PD-L1 expression by IHC (PD-L1 SP263 assay, TPS score assessment), they could confirm that patients with DCB had higher PD-L1 expression on cancer cells than No DCB patients (median PD-L1 expression for patients with DCB was 2.5 (95% CI[0-12.5]) and 0 for patients with No DCB (95% CI [0-0]; FIG. 2B). This difference was mostly driven by patients having objective partial responses on their RECIST.1 target lesions (FIG. 8). This finding was in accordance with the results recently generated in the above mentioned randomized phase 3 trial of Nivolumab+Ipilimumab trial (tumor cell staining with the PharmDx 28-8 PD-L1 assay) in advanced MM naïve of systemic therapies. Also, inventors performed bulk RNA sequencing on baseline (i.e., before immunotherapy) tumor biopsies and found a number of genes that were differentially expressed in DCB vs No DCB patients. Gene set enrichment analysis (GSEA), with the “Hallmark” panels, identified 1 gene sets which were significantly differentially expressed in biopsies of patients with DCB (FIG. 2C). Seven gene sets were upregulated and 4 were repressed in tumor biopsies of patients with DCB. Genes significantly upregulated in patients with DCB pertained to pathways related to oxidative phosphorylation (OXPHOS) metabolism, interferon alpha (IFNα) pathway, interferon gamma (IFNγ) pathway and “allograft rejection” signature, suggesting an ongoing adaptive immune response in the tumor. Significantly suppressed pathways in tumors from patients with DCB (therefore relatively enriched in tumors primary resistant to treatment), were genes from the epithelial to inesenchymal transition (EMT) pathway, G2M checkpoint, E2F and MYC targets. Those three E2F, MYC and G2M pathways shared several downstream gene expressions.

The result on Epithelial-Mesenchymal Transition (EMT) genes upregulation in patients with No DCB is in accordance with the above mentioned second biomarker recently reported to be associated to atezolizumab+bevacizumab resistance in refractory peritoneal MM. Beyond these reported predictive biomarkers, inventors found additional ones in the tumor and the blood by exploring fresh tumor biopsies and fresh whole blood.

First, flow cytometry analysis of fresh tumor biopsies at baseline found significantly more CD45+, CD3+ and CD3+CD8+ T-cells in patients with DCB (FIG. 2D). Comparatively, IHC estimation of T cells, B cells and macrophage tumor infiltrations in screening biopsies could not detect significant differences between patients with or without DCB (FIG. 9A and below Table 5).

TABLE 5
Immuno-Histo-Chemistry scoring of baseline tumor
biopsies according to their DCB status.

| | DCB | No DCB | |
|---|---|---|---|
| | (n = 8) | (n = 12) | p |
| CD8⁺ score | | | |
| 0 | 4 (50%) | 4 (33%) | 0.49 |
| 1 | 3 (38%) | 4 (33%) | |
| 2 | 0 | 3 (38%) | |
| 3 | 1 (12%) | 1 (8.3%) | |
| CD3⁺ CD8⁺ score | | | |
| 0 | 3 (38%) | 4 (33%) | 0.83 |
| 1 | 3 (38%) | 5 (42%) | |
| 2 | 2 (25%) | 2 (17%) | |
| 3 | 0 | 1 (8.3%) | |
| CD20⁺ score | | | |
| 0 | 6 (75%) | 6 (50%) | 0.5 |
| 1 | 1 (12%) | 4 (33%) | |
| 2 | 0 | 0 | |
| 3 | 1 (12%) | 2 (17%) | |
| TLS score | | | |
| 0 | 7 (88%) | 10 (83%) | 0.68 |
| 1 | 1 (12%) | 1 (8.3%) | |
| 2 | 0 | 1 (8.3%) | |

The test used to compare nominal measurements (semi quantitative scores) was χ² test.

Of note, the proportion of PD-1 positive CD4+ or CD8+ T-cells was not significantly higher in patients with DCB but there was a trend toward more PD1+ CD8+ T-cells in patients subsequently developing partial responses (FIG. 9B).

Flow cytometry analysis of fresh whole blood identified increased levels of Th1 T-cells in DCB patients, defined as CD3+CD4+ T-cells expressing CXCR3, the receptor of CXCL9 and CXCL10 (FIG. 2E). More specifically, patients with DCB presented higher levels of circulating effector memory T-cells expressing selectins (CLA) and integrins (a4b7, CD49a) which are typically prone to home into tissues (FIG. 2F).

Altogether, patients with DCB presented baseline features in their tumor and blood compatible with an ongoing anti-tumor T-cell activity, which was expected to pave the way to a response to checkpoint blockade immunotherapy.

### Pharmacodynamic Markers

Inventors took advantage of the one-week lead-in nintedanib monotherapy prior to the pembrolizumab introduction to analyze the respective effects of the two drugs. Sequential blood sampling was performed at baseline and on-treatment in order to identify selective phannacodynamic markers of Nintedanib and Pembrolizumab that occurred upon treatment in all patients and independently from the treatment efficacy. Plasma was obtained from blood draws collected at baseline (D-7), one week after the nintedanib monotherapy lead-in (C1D1) and one week after the addition of pembrolizumab (C1D8). First, inventors found that nintedanib monotherapy significantly decreased the levels of blood Angiopoitin-2, CCL21 and CCL23 in patients with both DCB and No DC B (FIG. 3A). CCL15 and CXCL10 were also decreasing in all patients upon Nintedanib monotherapy but this decrease was only significant for patients with DCB (FIG. 10A). Of note, CCL8 significantly increased during this first week of nintedanib in patients with No DCB, but tended to decrease in patients with DCB, suggesting potential opposite effects of anti-angiogenics on CCL8 in these two patient categories (FIG. 10B).

The addition of Pembrolizumab at C1D1 had a rapid impact on blood levels of soluble PD-1 (sPD1) and soluble PD-L1 (sPDL1). Although circulating sPD1 levels were undetectable at baseline in all patients at C1D1, they reached median levels above 10 ng/mL in patients with both DCB and No DCB after a week (C1D8, FIG. 3B). Conversely, inventors found detectable circulating levels of sPDL1 at baseline, which also significantly increased upon Pembrolizumab therapy in all patients after one week (FIG. 3C). As opposed to the decrease of CCL21 found upon Nintedanib lead-in monotherapy (FIG. 3A, middle), inventors found an increase in CCL19 in all patients after one week of combination with Pembrolizumab, illustrating potential opposite impacts of the combination therapy on ligands of CCR7, a chemokine receptor expressed by memory T-cells (FIG. 3D). Of note, CCL3, CCL8, CCL17 and CCL23 also increased while CXCL2 levels decreased upon Pembrolizumab addition but this was only significant in patients with No DCB (FIG. 10C).

Most surprisingly was the significant rise in CXCL9, CXCL0, CXCL11, and CXCL13 serum levels, which are potent Th1 and follicular T helper cell chemokines respectively, just one week after initiation of Pembrolizumab in patients with D1CB but also with No DCB (FIG. 3E).

Active Recruitment of CD8+ T-Cells Upon Treatment in the Tumors of Patients with No Durable Clinical Benefit

Patients prospectively underwent image-guided tumor biopsies at baseline and on-treatment (C2D1 prior second infusion of penbrolizumab). Fresh tumor biopsies were immediately put into physiological serum. Each tumor secretome (i.e., tumor biopsy supernatant) was subsequently collected for titration of cytokines and soluble factors released by the tumor tissue. Also, fresh tumor biopsies were turned into cell suspensions upon mechanic dissociation for flow cytometry (see FIG. 2A and Methods).

In order to check if the elevated blood chemokines detected at C1D8 were also secreted by tumors and maintained 3 weeks after combination treatment initiation, inventors analyzed the supernatant of the fresh tumor biopsies collected at C21. Within all the cytokines and chemokines titrated in the tumor biopsy supernatants, they found that CXCL9 was the most significant secreted cytokine by tumors between C2D1 and baseline (FIG. 4A). Indeed, they found a dramatic increase (˜x10) of CXCL9 secretion by tumor biopsies between baseline and on-treatment in patients with both DCB and No DCB but with higher values in the No DCB secretome (FIG. 4B).

Inventors wondered if those high levels of chemokine secretion would recruit T-cells in the tumors. Analyzing the dynamics of T-cell infiltrates from the flow cytometry data collected from paired fresh tumor biopsies at baseline and at C2D1 (see gating strategy in FIG. 11), they found indeed a significant increase in CD45+ (FIG. 4C, left), C1D3+ (FIG. 4D, left) and CD3+CD8+ (FIG. 4E, left) T-cells but predominantly in the tumors of patients with No DCB, who had very little T-cell infiltrates at baseline (FIG. 2D). The recruitment of those immune cells into tumors of patients with No DCB was also confirmed by absolute numbers of cells per biopsy by flow cytometry (FIGS. 4C/D/E, right). The analysis of RNAseq data from tumor biopsies also found an upregulation of CD8A, CD3e and CD3z gene expression which was only significant in the patients with No DCB (FIG. 4F and FIG. 12A). Interestingly, this T-cell fingerprint was associated with an upregulation of transcripts encoding for EOMES and T-bet transcription factors (FIGS. 4G and 4H respectively), gamma interferon (FIG. 12B) Granulysin (FIG. 4I), Granzyme A and B (FIG. 4J), which all characterize cytotoxic effector T-cells.

Unexpectedly, this active T-cell recruitment in tumors from patients with No DCB led to tumor CD8+ T-cell infiltrates that were equivalent to patients with DC B within only 3 weeks of combination treatment as they could not find any more differences in CD45+, CD3+ and CD3+CD8+ T-cell infiltrates between patients with DCB and No DCB at C2D1 (FIG. 4K).

Of note, immune infiltration in tumor analyses with deconvolution of bulk RNA-seq data highlighted that score for T cells, CD8 T cells, NK cells and cytotoxicity increased significantly between screening and C2D1 biopsies in No DCB patients (FIG. 12C). Moreover, semi-quantitative scoring of CD8 by IHC were not able to recapitulate accurately the flow cytometry and transcriptomic findings, although a trend toward increased densities of CD8+ cells was found in tumors with objective radiological responses upon combination treatment (FIGS. 9 and 12D).

Overall, inventors found that, upon pembrolizumab+nintedanib treatment, tumors from patients with No DCB secreted T-cell attracting chemokines and actively recruited CD8+ T-cells (more particularly CD45+CD3+CD8+ T cells) to a point that they were similar to tumors from patients with DCB only 3 weeks after C1D1.

Primary Resistance to Anti-PD1+Anti-Angiogenic is Associated with an Increase in Intratumoral Activated Tregs

In order to understand what was driving the resistance to anti-PD1+anti-angiogenics, inventors further explored the blood and tumor samples prospectively collected from the mesothelioma patients treated with pembrolizumab and nintedanib. First, they found that tumors from patients with No DCB presented with a significant increase in CD4+ T-cells proportions upon treatment (FIG. 5A), resulting in more CD4+ T-cells at C2D1 in tumors from patients with No DCB (FIG. 5B). Within those intratumoral CD4+ T-cells, they found significantly higher proportions of CD25 expressing cells at C2D1 (FIG. 5C) although no difference in CD4+CD25+ T-cells was found in biopsies at baseline (FIG. 12E). The dynamics of CD4+CD25+ numbers was indeed increasing in tumor biopsies of patients with No DCB (FIG. 5D). Concomitantly, soluble CD25 in secretome increased between screening and C2D1 in patients with No DCB as opposed to patients with DCB (FIG. 5E). Looking at the gene expression within those tumor biopsies, inventors could also find higher TIGIT expression in patients with No DCB upon treatment (FIG. 5F), together with ICOS (FIG. 5G), CTLA4 (FIG. 5H) and FOXP3 (FIG. 5) upregulation.

Altogether, these observations suggest that upon pembrolizumab and nintedanib treatment, mesothelioma patients with No DCB3 present more intraturnoral CD4+ T-cells with a phenotype compatible with activated regulatory T-cells (in particular CD45+CD3+CD8+ T cells).

Primary Resistance to Anti-PD1+Anti-Angiogenic is Associated with High VEGF, IL-8 and IL-6

In order to understand why, upon CXCL9 release, the recruitment of T-cells into tumors of patients with No DC B was in favor of CD4+ T-cells with high CD25 expression and FOXP3 transcription, inventors looked for factors that have been described to sustain Treg expansion. First, they found that VEGF-A was increasing in the secretome of the tumor biopsies from patients with No DC B (FIG. 6A). This resulted in significantly more VEGF-A at C2D1 in the secretome of the tumor biopsies from patients with No DCB (FIG. 6B). Interestingly, this trend for VEGF-A was not found in the blood (FIG. 12F). However, a similar trend was found in the blood for VEGF-D which was higher at baseline in the plasma of patients with No DC B (FIG. 6C) and tended to increase upon treatment in patients with No DC B while decreasing in patients with DC B (FIG. 6D).

In order to understand what could support the secretion of VEGF molecules in patients with No DCB, they titrated the levels of CXCL8 (interleukin-8) in the plasma of their patients and found significantly more circulating CXCL8 at baseline in patients with No DCI (FIG. 6E). Although CXCL8 levels remained stable and high upon treatment in the plasma of patients with No DCB (FIG. 6F), they tended to increase in their secretome (FIG. 6G). This resulted in significantly more CXCL8 in the secretome of patients with No DCB at C2D1 (FIG. 611). Inventors also titrated interleukin-6 (IL-6) because it has been frequently associated with CXCL8 expression and Treg development. They found high blood levels of interleukin-6 (IL-6) at baseline (D-7; FIG. 61). Those levels remained high upon the addition of nintedanib (C1D1; FIG. 6J) and pembrolizumab (C1D8; FIG. 6K). Inventors found that one of the potential sources of IL-6 in those patients was the tumor itself. Indeed, the concentrations in IL-6 were high in the secretome of tumors from patients with No DC B with a tendency to increase between baseline and C2D1 (FIG. 6L) resulting into significantly more IL-6 at C2D1 (FIG. 6M).

Overall, those results illustrate that mesothelioma patients with No DCB have a systemic pro-inflammatory and pro-angiogenic profile at baseline with increased tumor secretion of IL-6, CXCL8 and VEGF upon pembrolizumab and nintedanib therapy.

Genomic Somatic Copy Number Alterations Correlate with Plasma Interleukin-6 Levels

In order to better understand the biological characteristics of mesotheliomas from patients refractory to anti-PD1 and anti-angiogenic therapy, inventors decided to analyze the oncogenetic profiles of the tumors collected during the trial.

As differential tumor gene expression analyses from RNA-seq at baseline identified that the E2F pathway was enriched in patients with No DCB, inventors looked for chromosome 9 alterations by Fluorescent In Situ Hybridization (FISH), considering that 9p21 homozygous deletions, are frequently described in mesothelioma and leading to CDKN2A deletion. They found indeed that patients with No DCB presented twice as more chromosome 9 alterations (homozygous deletions, monosony, and heterozygous deletions) than patients with DCB (FIG. 7A/B).

Then, inventors performed whole exome sequencing (WES) from tumors collected during the trial in order to better understand the oncogenetic alterations found in their patients. Only 13 tumor biopsies were contributive (enough tumor material and sufficient proportions of cancer cells). They identified high numbers of somatic genomic alterations in patients with No DCB (FIG. 7C). Losses of tumor suppressor genes by point mutations and/or somatic copy number alterations (SCNA) were identified in BAPI (67%), EP300 (67%), NF2 (61%), SETD2 (61%), CDKN2A (56%), CREBBP (44%), TP53 (39%), MGA (22%) and DDX3X (17%) genes. The median tumor mutational burden was 0.7 mutations per megabase (0.48 to 2.11).

Inventors identified that patients with No DCB had a higher somatic copy number alteration score (SCNA score) than patients with DCB (FIG. 7D). Interestingly, they found a positive correlation between the number of SCNA and IL-6 plasma levels (FIG. 7E).

When integrating the different biomarkers described above into a single heatruap, they found indeed higher values of SCNA with tumor and blood biomarkers associated with No DCB (FIG. 7F). Of note, the most consistent predictive biomarker for DCB was the proportion of CD8+ cells among the total CD3+ cells, in particular among the CD45+CD3+ T-cells, as assessed by flow cytometry on baseline fresh tumor biopsies (FIGS. 2D and 7F).

### Discussion

Mesothelioma is one of the latest tumor indication where the benefits of immunotherapy targeting the PD-1/PD-L1 pathway has been demonstrated. Here, inventors show that the combination of pembrolizumab 200 mg Q3W with nintedanib 150 mg BID provides significant antitumor activities and manageable toxicities in patients with advanced (pleural) mesothelioma naïve to immunotherapy and refractory/to first line of platinum-based chemotherapy. Of note, pembrolizumab monotherapy in this patient population generated a best objective response rate (BORR) of 8% in the Keynote-158 study per RECIST1.1 (Yap T A et al.). Here, the addition of the anti-angiogenic tyrosine kinase inhibitor nintedanib to pembrolizumab compared favorably to this historical dataset with a three-fold higher BORR of 24% using the same radiological criteria, although in a smaller patient population (n=30 vs n=118). More recently, the combination of an intra-pleural anti-mesothelin CAR-T cell therapy with pembrolizumab generated a BORR of 12.5% in the same patient population but with mRECIST criteria (Adusumilli P S et al.).

Beyond clinical outcomes, the extensive biological explorations performed in this study shed light on the mechanisms associated with primary resistance of pleural mesotheliomas to anti PD-(L)1 immunotherapy combined with antiangiogenic agents, in particular antiangiogenic TKI. First, inventors confirmed that a statistically significant higher expression of PD-L1 by IHC on cancer cells and that an epithelial rather than mesenchymal gene expression signature are found in mesothelioma tumors of patients having a better outcome under PD-1/PD-L1 targeted immunotherapies (Raghav K. et al.; Baas P. et al.). However, such biomarkers are poorly sensitive and specific at the individual level because of a broad overlap of values between patients with favorable and bad outcomes. The use of flow cytometry on fresh samples at baseline identified new and potentially more robust predictive biomarkers of outcome upon anti-PD1 or anti-PD-L1 immunotherapy in mesothelioma which are the proportion of CD8+ T-cells within the total tumor infiltrating CD3+ T-cells, in particular within tumor infiltrating CD45+CD3+ T cells, and high levels of effector memory T-cells in the blood expressing the selectin CLA, the integrins a4b7 and CD49a, and/or the chemokine receptor CXCR3. The 7 days monotherapy lead-in allowed us to describe that the immediate pharmacodynamic effects of nintedanib were to diminish the circulating levels of Angiopoietin-2, CCL21 and CCL23, which are growth factors and pro-inflammatory chemokines known to be associated with mesothelioma pathogenesis (Magkouta S. et al.; hang Q. et al.; Ostroff R M et al). The addition of pembrolizumab increased dramatically in only one week the blood levels of CXCL9 and CXCL10 which are both potent T-cell attractants (known pharmacodynamic effects of anti-PD1 therapies—cf. Letourneur D. et al.). Here, inventors show that this effect also occurred in tumors, where high levels of CXCL9 were detected in the secretome of all tumor biopsies 3 weeks after C1D1. Surprisingly, this CXCL9 burst was associated with a strong T-cell recruitment into tumors of patients with No DCB, notably CD8+ T-cells with a cytotoxic phenotype, which reached a proportion comparable to tumors of patients with DCB after only one cycle of treatment. The difference observed in tumor biopsies of the two subsets of patients was mostly regarding the CD4+ T-cell composition of those immune infiltrates. Tumors from patients with No DCB presented a large proportion of CD4+ T-cells which presented features of activation compatible with an immunosuppressive regulatory T-cell (Treg) phenotype. One hypothesis which could explain why the phenotype of tumor infiltrating T-cells was skewed toward Tregs in tumors from patients with No DCB would be the pre-existing pro-inflammatory and pro-angiogenic contexture of those tumors. Indeed, patients with No DCB presented high levels of VEGF, CXCL8 (IL-8) and IL-6 in both their blood and tumor secretome at baseline compared to patients with DCB, and those levels were even increasing upon treatment. The VEGF pathways have been recently described as key pathological features of human mesothelioma (Alcala N. et al.). A number of publications suggest that, across tumor types, VEGF molecules may play a role beyond angiogenesis and support the immunosuppression within the tumor micro-environment via their direct effects on CD8+ T-cells (Voron T. et al.), dendritic cells (Gabrilovich D I et al.), and Tregs (Li B. et al.; Terme M. et al.). In mesothehoma VEGF, CXCL8 and IL-6 are well known pro-tumoral and autocrine factors (Abdul Rahim S N et al.; Nowak A K et al.; Galffy G. et al.; Donnenberg A D et al.). Interestingly, IL-6 has been described to induce proliferation and VEGF expression in mesothelioma cancer cell lines (Adachi Y. et al.). Although inventors could not find significant differences between subgroups of patients in terms of M1 or M2 macrophages according to CD68 and CD163 expression by IHC on tumor biopsies, they believe, without wishing to be bound by any particular theory, that it is possible that the phenotype of tumor infiltrative myeloid cells are playing an important role as they are believed to be major sources of IL-6, IL-8 and VEGF in the tumor microenvironment. Very surprising is also inventors' finding of a correlation between somatic copy number alterations and circulating levels of IL6. Some of the frequent somatic genomic alterations found in mesothelioma might indeed sustain the pro-inflammatory and pro-angiogenic phenotype of those tumors. For instance, TP53 mutations, frequently found in our patients with No DCB and typical genomic alterations of mesothelioma (Yap T A et al.), have been shown to correlate with higher secretions of VEGF-A in non-small cell lung cancers (Schwaederld M. et al.). Another frequent genomic alteration in mesothelioma that inventors found frequent in their patients with No DCB concerned the chromosome 9, notably the 9p21 chromosomal region which contains the CDKN2A gene. CDKN2A is also a classical genomic alteration of mesothelioma (Yap T A et al.). However, what is less known is the usual loss of type I interferon genes together with CDKN2A losses (Grard M. et al.). Interestingly, those CDKN2A associated type I interferon losses have been shown to present altered immune gene signatures in tumors (Peng Y. et al.). In myeloma cancer cell lines, which are also frequently depending on IL-6 autocrine signaling, it has been shown that the addition of IFNα in the culture media can downregulate the gene and protein expression of IL-6 receptor subunits and therefore directly hamper the positive feedback loops of the pathway (Schwabe M. et al.). Inventors believe that the loss of type I IFN expression in mesothelioma via 9p21 alterations could therefore support the autocrine IL-6 growth signaling by absence of downregulation of IL-6 receptor subunits.

### CONCLUSION

Sequential explorations of fresh tumor biopsies demonstrated that mesothelioma resistance to anti-PD+anti-angiogenics is not due to a lack of tumor T cell infiltration but rather to adaptive immunosuppressive pathways by tumors involving molecules (e.g., IL-6, CXCL8, VEGF, CTLA4, etc.) that are amenable to targeted therapies.

Overall, inventors found that mesothelioma patients having primary resistance to PD-1 or PD-L1 targeted immunotherapy and anti-angiogenesis agents were actively recruiting CD45+CD3+CD8+ cytotoxic T-cells in their tumors through CXCL9 tumor release upon treatment, and displayed high VEGF (A in the secretome & D in the blood), CXCL8 and/or IL-6 levels both in the blood and the tumor, and those correlated with high genomic somatic copy number alterations in the tumor (somatic) genome.

The herein described biological characterization of mesothelioma patients now advantageously allows a better stratification of patients' clinical care and offer a biomarker driven—rather than simple histology-driven stratification of therapeutic strategies.

### REFERENCES

Abdul Rahim S N, Ho G Y, Coward JIG. The role of interleukin-6 in malignant mesothelioma. Transl Lung Cancer Res. 2015; 4:55-66.
Adachi Y, Aoki C, Yoshio-Hoshino N, Takayama K, Curiel D T, Nishimoto N. Interleukin-6 induces both cell growth and VEGF production in malignant mesotheliomas. Int J Cancer. 2006; 119:1303-11.
Adusumilli P S, Zauderer M G, Rivibre I, Solomon S B, Rusch V W, O'Cearbhaill R E, et al. A Phase I Trial of Regional Mesothelin-Targeted CAR T-cell Therapy in Patients with Malignant Pleural Disease, in Combination with the Anti-PD-1 Agent Pembrolizumab. Cancer Discov. 2021; 11:2748-63.
Alcala N, Mangiante L, Le-Stang N, Gustafson C E, Boyault S, Damiola F, et al. Redefining malignant pleural mesothelioma types as a continuum uncovers immune-vascular interactions. EBioMedicine. 2019; 48:191-202.
Baas P, Scherpereel A, Nowak A K, Fujimoto N, Peters S, Tsao A S, et al. First-line nivolumab plus ipilimumab in unresectable malignant pleural mesothelioma (CheckMate 743): a multicentre, randomised, open-label, phase 3 trial. Lancet. 2021; 397:375-86.
Baldini C, Danlos F-X, Varga A, Texier M, Halse H, Mouraud S, et al. Safety, recommended dose, efficacy and immune correlates for nintedanib in combination with pembrolizumab in patients with advanced cancers. J Exp Clin Cancer Res. 2022; 41:217.
Danlos, F.-X., Texier, M., Job, B., Mouraud, S., Cassard, L., Baldini, C., Varga, A., Yurchenko, A. A., Rabeau, A., Champiat, S., et al. (2023). Genomic Instability and Protumoral Inflammation Are Associated with Primary Resistance to Anti-PD-1+Antiangiogenesis in Malignant Pleural Mesothelioma. Cancer Discov. 13, 858-879.
DePristo M A, Banks E, Poplin R, Garimella K V, Maguire J R, Hartl C, et al. A framework for variation discovery and genotyping using next-generation DNA sequencing data. Nat Genet. 2011; 43:491-8.
Donnenberg A D, Luketich J D, Donnenberg V S. Secretome of pleural effusions associated with non-small cell lung cancer (NSCLC) and malignant mesothelioma: therapeutic implications. Oncotarget. 2019; 10:6456-65.
Dostert C, Pétrilli V, Van Bruggen R, Steele C, Mossman B T, Tschopp J. Innate immune activation through Nalp3 inflammasome sensing of asbestos and silica. Science. 2008; 320:674-7.
Dubuisson, A., Fahrmer, J.-E., Goubet, A.-G., Terrisse, S., Voisin, N., Bayard, C., Lofek, S., Drubay, D., Bredel, D., Mouraud, S., et al. (2021). Immunodynamics of explanted human tumors for immuno-oncology. EMBO Mol. Med. 13, e12850.
Ewels P, Magnusson M, Lundin S, Käller M. MultiQC: summarize analysis results for multiple tools and samples in a single report. Bioinformatics. 2016; 32:3047-8.
Finn R S, Qin S, Ikeda M, Galle P R, Ducreux M, Kim T-Y, et al. Atezolizumab plus Bevacizumab in Unresectable Hepatocellular Carcinoma. N Engl J Med. 2020; 382:1894-905.
Forde P M, Anagnostou V, Sun Z, Dahlberg S E, Kindler H L, Niknafs N, et al. Durvalumab with platinum-pemetrexed for unresectable pleural mesothelioma: survival, genomic and immunologic analyses from the phase 2 PrE0505 trial. Nat Med. 2021; 27:1910-20.
Gabrilovich D I, Chen H L, Girgis K R, Cunningham H T, Meny G M, Nadaf S, et al. Production of vascular endothelial growth factor by human tumors inhibits the functional maturation of dendritic cells. Nat Med. 1996; 2:1096-103.
Galateau-Salle F, Churg A, Roggli V, Travis W D, World Health Organization Committee for Tumors of the Pleura. The 2015 World Health Organization Classification of Tumors of the Pleura: Advances since the 2004 Classification. J Thorac Oncol. 2016; 11:142-54.
Galffy G, Mohammed K A, Dowling P A, Nasreen N, Ward M J, Antony V B. Interleukin 8: an autocrine growth factor for malignant mesothelioma. Cancer Res. 1999; 59:367-71.
Grard M, Chatelain C, Delaunay T, Pons-Tostivint E, Bennouna J, Fonteneau J-F. Homozygous Co-Deletion of Type I Interferons and CDKN2A Genes in Thoracic Cancers: Potential Consequences for Therapy. Front Oncol. 2021; 11:695770.
Grosso F, Steele N, Novello S, Nowak A K, Popat S, Greillier L, et al. Nintedanib Plus Pemetrexed/Cisplatin in Patients With Malignant Pleural Mesothelioma: Phase II Results From the Randomized, Placebo-Controlled LUME-Meso Trial. J Clin Oncol. 2017; 35:3591-600.
Grothey A, Galanis E. Targeting angiogenesis: progress with anti-VEGF treatment with large molecules. Nat Rev Clin Oncol. 2009; 6:507-18.
Jiang Q, Ghafoor A, Mian I, Rathkey D, Thomas A, Alewine C, et al. Enhanced efficacy of mesothelin-targeted immunotoxin LMB-100 and anti-PD-1 antibody in patients with mesothelioma and mouse tumor models. Sci Transl Med. 2020; 12.
Koster J, Rahmann S. Snakemake-a scalable bioinformatics workflow engine. Bioinformatics. 2018; 34:3600.
Letourneur D, Danlos F-X, Marabelle A. Chemokine biology on immune checkpoint-targeted therapies. Eur J Cancer. 2020; 137:260-71.
Li H, Durbin R. Fast and accurate short read alignment with Burrows-Wheeler transform. Bioinformatics. 2009; 25:1754-60.
Li H, Handsaker B, Wysoker A, Fennell T, Ruan J, Homer N, et al. The Sequence Alignment/Map format and SAMtools. Bioinformatics. 2009; 25:2078-9.
Li B, Lalani A S, Harding T C, Luan B, Koprivnikar K, Huan Tu G, et al. Vascular endothelial growth factor blockade reduces intratumoral regulatory T cells and enhances the efficacy of a GM-CSF-secreting cancer immunotherapy. Clin Cancer Res. 2006; 12:6808-16.
Love M I, Huber W, Anders S. Moderated estimation of fold change and dispersion for RNA-seq data with DESeq2. Genome Biol. 2014; 15:550.
Love M I, Soneson C, Patro R. Swimming downstream: statistical analysis of differential transcript usage following Salmon quantification. F1000Res. 2018; 7:952.
Magkouta S, Kollintza A, Moschos C, Spella M, Skianis I, Pappas A, et al. Role of angiopoietins in mesothelioma progression. Cytokine. 2019; 118:99-106.
Mayakonda A, Lin D-C, Assenov Y, Plass C, Koeffler H P. Maftools: efficient and comprehensive analysis of somatic variants in cancer. Genome Res. 2018; 28:1747-56.
Merino D M, McShane L M, Fabrizio D, Funari V, Chen S-J, White J R, et al. Establishing guidelines to harmonize tumor mutational burden (TMB): in silico assessment of variation in TMB quantification across diagnostic platforms: phase I of the Friends of Cancer Research TMB Harmonization Project. J Immunother Cancer. 2020; 8:e000147.
Morgan M, Pages H, Obenchain V, Hayden N. Rsamtools: Binary alignment (BAM), FASTA, variant call (BCF), and tabix file import [Internet]. Bioconductor version: Release (3.13); 2021 [cited 2021 Sep. 1]. Available from: https://bioconductor.org/packages/Rsamtools/
Musk A W, Olsen N, Alfonso H, Reid A, Mina R, Franklin P, et al. Predicting survival in malignant mesothelioma. Eur Respir J. 2011; 38:1420-4.
Nowak A K, Brosseau S, Cook A, Zalcman G. Antiangiogeneic Strategies in Mesothelioma. Front Oncol. 2020; 10:126.
Nowak A K, Lesterhuis W J, Kok P-S, Brown C, Hughes B G, Karikios D J, et al. Durvalumab with first-line chemotherapy in previously untreated malignant pleural mesothelioma (DREAM): a multicentre, single-arm, phase 2 trial with a safety run-in. Lancet Oncol. 2020; 21:1213-23.
Ostroff R M, Mehan M R, Stewart A, Ayers D, Brody E N, Williams S A, et al. Early detection of malignant pleural mesothelioma in asbestos-exposed individuals with a noninvasive proteomics-based surveillance tool. PLoS One. 2012; 7:e46091.
Patro R, Duggal G, Love M I, Irizarry R A, Kingsford C. Salmon provides fast and bias-aware quantification of transcript expression. Nat Methods. 2017; 14:417-9.
Peng Y, Chen Y, Song M, Zhang X, Li P, Yu X, et al. Co-occurrence of CDKN2A/B and IFN-I homozygous deletions correlates with an immunosuppressive phenotype and poor prognosis in lung adenocarcinoma. Mol Oncol. 2022; 16:1746-60.
Pitoiset F, Cassard L, El Soufi K, Boselli L, Grivel J, Roux A, et al. Deep phenotyping of immune cell populations by optimized and standardized flow cytometry analyses. Cytometry A. 2018; 93:793-802.
Raghav K, Liu S, Overman M J, Willett A F, Knafi M, Fu S-C, et al. Efficacy, Safety and Biomarker Analysis of Combined PD-L1 (Atezolizumab) and VEGF (Bevacizumab) Blockade in Advanced Malignant Peritoneal Mesothelioma. Cancer Discov. 2021; candisc.0331.2021.
Ramos A H, Lichtenstein L, Gupta M, Lawrence M S, Pugh T J, Saksena G, et al. Oncotator: cancer variant annotation tool. Hum Mutat. 2015; 36:E2423-2429.
Ribas A, Wolchok J D. Cancer immunotherapy using checkpoint blockade. Science. 2018; 359:1350-5.
Rini B I, Plimack E R, Stus V, Gafanov R, Hawkins R, Nosov D, et al. Pembrolizumab plus Axitinib versus Sunitinib for Advanced Renal-Cell Carcinoma. N Engl J Med. 2019; 380:1116-27.
Scagliotti G V, Gaafar R, Nowak A K, Nakano T, van Meerbeeck J, Popat S, et al. Nintedanib in combination with pemetrexed and cisplatin for chemotherapy-naive patients with advanced malignant pleural mesothelioma (LUME-Meso): a double-blind, randomised, placebo-controlled phase 3 trial. Lancet Respir Med. 2019; 7:569-80.
Schwabe M, Brini A T, Bosco M C, Rubboli F, Egawa M, Zhao J, et al. Disruption by interferon-alpha of an autocrine interleukin-6 growth loop in IL-6-dependent U266 myeloma cells by homologous and heterologous down-regulation of the IL-6 receptor alpha- and beta-chains. J Clin Invest. 1994; 94:2317-25.
Schwaederld M, Lazar V, Validire P, Hansson J, Lacroix L, Soria J-C, et al. VEGF-A Expression Correlates with TP53 Mutations in Non-Small Cell Lung Cancer: Implications for Antiangiogenesis Therapy. Cancer Res. 2015; 75:1187-90.
Schwartz L H, Litiére S, de Vries E, Ford R, Gwyther S, Mandrekar S, et al. RECIST 1.1-Update and clarification: From the RECIST committee. Eur J Cancer. 2016; 62:132-7.
Socinski M A, Jotte R M, Cappuzzo F, Orlandi F, Stroyakovskiy D, Nogami N, et al. Atezolizumab for First-Line Treatment of Metastatic Nonsquamous NSCLC. N Engl J Med. 2018; 378:2288-301.
Sturm G, Finotello F, Petitprez F, Zhang J D, Baumbach J, Fridman W H, et al. Comprehensive evaluation of transcriptome-based cell-type quantification methods for immuno-oncology. Bioinformatics. 2019; 35:i436-45.
Terme M, Pernot S, Marcheteau E, Sandoval F, Benhamouda N, Colussi O, et al. VEGFA-VEGFR pathway blockade inhibits tumor-induced regulatory T-cell proliferation in colorectal cancer. Cancer Res. 2013; 73:539-49.
Van der Auwera G A, Carneiro M O, Hartl C, Poplin R, Del Angel G, Levy-Moonshine A, et al. From FastQ data to high confidence variant calls: the Genome Analysis Toolkit best practices pipeline. Curr Protoc Bioinformatics. 2013; 43:11.10.1-11.10.33.
Van Loo P, Nordgard S H, Lingjxrde O C, Russnes H G, Rye I H, Sun W, et al. Allele-specific copy number analysis of tumors. Proc Natl Acad Sci USA. 2010; 107:16910-5.
Vogelzang N J, Rusthoven J J, Symanowski J, Denham C, Kaukel E, Ruffie P, et al. Phase III study of pemetrexed in combination with cisplatin versus cisplatin alone in patients with malignant pleural mesothelioma. J Clin Oncol. 2003; 21:2636-44.
Voron T, Colussi O, Marcheteau E, Pernot S, Nizard M, Pointet A-L, et al. VEGF-A modulates expression of inhibitory checkpoints on CD8+ T cells in tumors. J Exp Med. 2015; 212:139-48.
Wallin J J, Bendell J C, Funke R, Sznol M, Korski K, Jones S, et al. Atezolizumab in combination with bevacizumab enhances antigen-specific T-cell migration in metastatic renal cell carcinoma. Nat Commun. 2016; 7:12624.
Xu A, Wu L J, Santella R M, Hei T K. Role of oxyradicals in mutagenicity and DNA damage induced by crocidolite asbestos in mammalian cells. Cancer Res. 1999; 59:5922-6.
Yap T A, Aerts J G, Popat S, Fennell D A. Novel insights into mesothelioma biology and implications for therapy. Nat Rev Cancer. 2017; 17:475-88.
Yap T A, Nakagawa K, Fujimoto N, Kuribayashi K, Guren T K, Calabró L, et al. Efficacy and safety of pembrolizumab in patients with advanced mesothelioma in the open-label, single-arm, phase 2 KEYNOTE-158 study. Lancet Respir Med. 2021; 9:613-21.
Yu G, Wang L-G, Han Y, He Q-Y. clusterProfiler: an R package for comparing biological themes among gene clusters. OMICS. 2012; 16:284-7.
Zalcman G, Mazieres J, Margery J, Greillier L, Audigier-Valette C, Moro-Sibilot D, et al. Bevacizumab for newly diagnosed pleural mesothelioma in the Mesothelioma Avastin Cisplatin Pemetrexed Study (MAPS): a randomised, controlled, open-label, phase 3 trial. Lancet. 2016; 387:1405-14.

1. An in vitro or ex vivo method of predicting, assessing or monitoring the sensitivity or resistance of a subject having a cancer to a therapy combining i) an immunotherapeutic agent, in particular an anti-PD-1, or anti-PD-L1, monoclonal antibody, and ii) an anti-angiogenic agent, after one or several treatment steps in the subject, wherein the method comprises:
a step a) of determining in a tumor sample of the subject, the proportion of CD8+ T-cells among live CD45+CD3+ T cells, and a step b) of comparing said live CD8+CD3+CD45+ T cells proportion to a CD8+CD3+CD45+ T cells reference proportion, a proportion of live CD8+CD3+CD45+ T cells below (<) the CD8+CD3+CD45+ T cells reference proportion being indicative of resistance of the subject to the combination therapy and a proportion of live CD8+CD3+CD45+ T cells superior or equal to (≥) the CD8+CD3+CD45+ T cells reference proportion being indicative of sensitivity of the subject to the combination therapy;
a step a) of determining in a blood sample of the subject, the expression level of effector memory (EM) CD4+ T cells expressing the alpha-4 beta-7 integrin (a4b7+ EM CD4+ T cells), cutaneous lymphocyte antigen selectin (CLA+ EM CD4+ T cells), CD49a integrin (CD49a+EM CD4+ T cells) and/or CXCR3 chemokine receptor (CXCR3+ Th1 CD4+ T cells), and a step b) of comparing said expression level(s) to a4b7+ EM CD4+ T cells, CLA+ EM CD4+ T cells, CD49a+ EM CD4+ T cells and/or CXCR3+ Th1 CD4+ T cells reference expression level(s), (an) expression level(s) superior or equal to (≥) the reference expression level(s) being indicative of sensitivity of the subject to the combination therapy, and (an) expression level(s) below (<) the reference expression level(s) being indicative of resistance of the subject to the combination therapy;
a step a) of determining in a blood plasma sample or in a tumor supernatant sample of the subject, the concentration of VEGFA, VEGFD, CXCL8 and/or IL6 protein(s), and a step b) of comparing said concentration(s) to VEGFA, VEGFD, CXCL8 and/or IL6 protein(s) reference concentration(s), concentration(s) superior or equal to (≥) the reference concentration(s) being indicative of resistance of the subject to the combination therapy, and concentration(s) below (<) the reference concentration(s) being indicative of sensitivity of the subject to the combination therapy; and/or
a step of determining in cancerous cells of a tumor sample of the subject, the number of somatic alterations, a somatic copy number alteration score or genomic instability score (“SCNA score”) above (>) a reference score being indicative of resistance of the subject to the combination therapy, and a SCNA score equal to or below (≤) the reference score being indicative of sensitivity of the subject to the combination therapy.

2. The method according to claim 1, wherein the proportion of CD8+ T cells among live CD45+CD3+ T cells reference is the proportion of CD8+ T cells among live CD45+CD3+ T cells in the tumor of the subject before any immunotherapeutic treatment step in the subject.

3. The method according to claim 1, wherein the anti-PD-1 monoclonal antibody is selected from pembrolizumab, nivolumab, cemiplimab and dostarlimab, or the anti-PD-L1 monoclonal antibody is selected from atezolizumab, durvalumab and avelumab.

4. The method according to claim 1, wherein the anti-angiogenic agent is an anti-VEGF agent, for example bevacizumab, and/or a tyrosine kinase inhibitor (TKI), for example nintedanib.

5. The method according to claim 1, wherein the cancer is a mesothelioma (MM).

6. The method according to claim 1, wherein the tumor sample is a fresh tumor sample or the tumor supernatant sample of a fresh tumor sample.

7. The method according to claim 6, wherein the tumor sample is obtained with a needle selected from a 16G to 20G needle for tumor biopsy, typically a 18G needle.

8. The method according to claim 6, wherein the fresh tumor sample is being put immediately in a volume between 200 μL and 1 mL of a preservation medium before step a) and/or before any collection of cancerous cells from the tumor sample.

9. The method according to claim 6, wherein the fresh tumor sample is dissociated with both enzymatic and mechanical procedures before being stained and used in the method.

10. The method according to claim 1, wherein the blood sample is a fresh whole blood sample.

11. The method according to claim 6, wherein the method comprises a step of dosing via ultrasensitive titration assay at least one marker selected from VEGFA, IL6, IL8 (CXCL8) and granzyme (GZMA and/or GZMB) in the supernatant of the tumor sample after an incubation step of said tumor sample of at least one minute, and up to 72 h, or a step of dosing at least one of said markers in the plasma obtained after double centrifugation of the fresh whole blood sample.

12. The method according to claim 10, wherein the method to analyse the phenotype of immune, stromal and/or cancer cells from the fresh tumor sample or fresh whole blood sample is based on conventional or spectral flow cytometry.

13. A method of selecting an appropriate therapeutic treatment for a subject having a cancer, which method comprises a step of predicting or assessing the sensitivity of a subject having a cancer to a cancer treatment combining an immunotherapeutic agent and an anti-angiogenic agent using a method according to claim 1.

14. A method of selecting or disqualifying a subject having a cancer for inclusion in a clinical trial, the clinical trial being for evaluating a cancer treatment combining an immunotherapeutic agent and an anti-angiogenic agent, which method comprises a step of predicting or assessing the sensitivity of a subject having a cancer to the combination therapy, using a method according to claim 1.

15. Use of a kit for predicting, assessing or monitoring the sensitivity of a subject having a tumor to a cancer treatment combining an anti-PD-1, or anti-PD-L1, monoclonal antibody and an anti-angiogenic agent, wherein the kit contains at least two distinct antibodies selected from an antibody recognizing a viability dye, CD45, CD8, CD3, a4b7, CLA, CD49a, CXCR3, CD45RO or CD45RA, CCR7, IL6, IL8 (CXCL8), VEGFA, VEGFD, Granzyme A, and Granzyme B, as detection means, and, optionally, a leaflet providing corresponding reference expression levels.
